(19) United States
(12) Patent Application Publication
(10) Pub. No.: US 2025/0344956 A1
(43) Pub. Date: Nov. 13, 2025

(54) RADIAL DISPLACEMENT PULSE WAVE MEASURING DEVICE AND APPLICATION METHOD THEREOF

(71) Applicant: Ming-Cheng Shih, Hsinchu (TW)

(72) Inventor: Ming-Cheng Shih, Hsinchu (TW)

(21) Appl. No.: 19/274,679

(22) Filed: Jul. 21, 2025

Related U.S. Application Data
(63) Continuation of application No. PCT/CN2023/127276, filed on Oct. 27, 2023.
Application No. PCT/CN2023/127276 is a continuation-in-part of application No. 18/098,729, filed on Jan. 19, 2023.
(63) Continuation-in-part of application No. 18/098,729, filed on Jan. 19, 2023.
(60) Provisional application No. 63/304,367, filed on Jan. 28, 2022.

(51) Int. Cl. A61B 5/021; A61B 5/00; A61B 5/022; A61B 5/0225; A61B 5/024; A61B 5/107; G16H 40/63

(52) U.S. Cl. CPC A61B 5/02125; A61B 5/02141; A61B 5/02233; A61B 5/0225; A61B 5/02405; A61B 5/107; A61B 5/6813; A61B 5/6844; A61B 5/7203; A61B 5/7264; G16H 40/63; A61B 2562/0233; A61B 2562/046

(57) ABSTRACT

A radial displacement pulse wave measuring device and application methods thereof are provided. The radial displacement pulse wave measuring device includes an airbag, a pressure control module, a displacement sensing module, and a computing unit. The pressure sensor of the pressure control module is configured to measure a vascular volumetric pulse wave of an artery, while the displacement sensing module is configured to measure a vascular radial displacement pulse wave by detecting dynamic distance variations between the displacement sensing module and a measure site covering the artery. The computing unit controls pressure adjustment of the airbag via the pressure control module and scanning via the displacement sensing module to optimize measurement conditions for accurately recording the vascular radial displacement pulse wave.

### CROSS-REFERENCE TO RELATED APPLICATION

The present application is a continuation application of PCT application No. PCT/CN2023/127276 filed on Oct. 27, 2023, which claims the benefit of U.S. patent application Ser. No. 18/098,729 filed on Jan. 19, 2023. This application is also a Continuation-in-part Application of U.S. patent application Ser. No. 18/098,729, filed on Jan. 19, 2023. U.S. patent application Ser. No. 18/098,729 claims the benefit of U.S. provisional Application No. 63/304,367 filed on Jan. 28, 2022. The entire contents of all of the aforementioned applications are incorporated by reference herein in their entirety.

### BACKGROUND

### Technical Field

The present disclosure relates to a physiological characteristic measurement system and a method for using the same, and more particularly, to a radial displacement pulse wave measuring device and a method for using the same.

### Description of Related Art

Vascular pulse waves are used to assess the function and health status of the cardiovascular system. Depending on the characteristics of the measurement, vascular pulse waves can be classified into pressure pulse waves, vascular volume pulse waves, photoplethysmography (PPG) pulse waves, and vascular radial displacement pulse waves.

The above-mentioned pressure pulse waves are typically measured using piezoelectric, strain gauge, film-type, or other piezoresistive pressure sensors. The pressure sensors indirectly measure the pressure caused by vascular expansion. In use, the pressure sensor is placed between an airbag and the skin. When the airbag, which is attached to the skin, expands, the airbag presses downward on the blood vessel located below the airbag. As the pulse wave changes the diameter of the blood vessel, the blood vessel compresses the pressure sensor and the airbag located above the blood vessel. Accordingly, the pressure sensor can indirectly measure the pressure pulse wave of the blood vessel based on the change in vessel diameter. Common problems with pulse wave measurement using this approach include a small dynamic measurement range, a low signal-to-noise ratio, complex calibration methods for the pressure sensor, and poor reproducibility of measurement results.

The above-mentioned vascular volume pulse waves are typically measured using a cuff-type airbag as a measurement tool, and a pressure sensor is used to measure the pressure inside the cuff-type airbag. In use, the cuff-type airbag is placed above a blood vessel, and the airbag is gradually inflated and pressurized. A pressure sensor then measures changes in the internal pressure of the airbag. The measurement result corresponds to integrating the diameter changes of all blood vessels located under the airbag and converting them into a change in the internal pressure of the airbag. A problem with measuring pulse waves using this approach is that when the air inside the airbag is compressed, the details of the vascular volume pulse wave disappear, leaving only the amplitude of the vascular volume pulse wave available for reference.

The above-mentioned photoplethysmography (PPG) pulse wave is obtained by using a PPG sensor to detect changes in the intensity of light reflected from blood vessels located beneath the skin. A common problem with measuring pulse waves using this approach is that physiological parameter differences at the measurement location of an individual (such as subcutaneous tissue thickness or tissue light absorption properties) may affect the measurement results of the PPG pulse wave.

The above-mentioned vascular radial displacement pulse wave can also be measured using ultrasonic echo imaging. However, complex computations and calibrations are often required to achieve good image resolution and contrast to estimate the amount of change in vascular radial displacement.

### SUMMARY

To address the above problems, one aspect of the present disclosure is to provide a radial displacement pulse wave measuring device for measuring a vascular radial displacement pulse wave of an artery at a measurement site of a subject. The radial displacement pulse wave measuring device comprises a housing, a transparent airbag, a pressure control module, a displacement sensing module, and a computing unit.

The housing has a transparent portion, and the transparent portion comprises a hole or a first transparent plate. A transparent airbag is disposed below the transparent portion, and a main material of the transparent airbag is a material that is resistant to stretching deformation. The pressure control module is configured to control an internal pressure of the transparent airbag to control a downward pressure applied by the transparent airbag to the measurement site to enhance a pulse signal of the artery. The displacement sensing module is disposed above the transparent portion. The displacement sensing module is configured to sense pulse beats of the artery and measure a dynamic distance between a skin of the measurement site and the displacement sensing module caused by the pulse beats of the artery to obtain the vascular radial displacement pulse wave. The computing unit is communicationally connected to the pressure control module and the displacement sensing module. The computing unit is configured to respectively transmit control signals to the pressure control module and the displacement sensing module, as well as respectively receive information transmitted from the pressure control module and the displacement sensing module for performing calculations.

According to one embodiment of the present disclosure, a main material of the transparent airbag comprises polymethyl methacrylate, cellulose acetate, nylon-66 polyamide resin, nylon-6 polyamide resin, polybutylene terephthalate, polyethylene terephthalate, polyphenylene oxide, polycarbonate, ethylene-vinyl acetate copolymer, polyethylene, polypropylene, polyvinyl chloride, polyoxymethylene, or polyurethane.

According to one embodiment of the present disclosure, when the transparent portion is the hole, the transparent airbag further comprises a transparent window overlapping the transparent portion of the housing. The transparent window comprises a second transparent plate.

According to one embodiment of the present disclosure, an anti-reflection coating is disposed on an outer surface of the transparent window.

According to one embodiment of the present disclosure, an area of the second transparent plate is larger than an area of the hole, such that when an internal pressure of the transparent airbag is excessive, the second transparent plate abuts against a lower side of the hole and is prevented from being pushed to an upper side of the hole.

According to one embodiment of the present disclosure, the transparent airbag further comprises a contact portion configured to closely fit the skin of the measurement site of the subject, and a material of the contact portion is a soft, stretchable, and deformable thermoplastic elastomer material, which comprises at least one of thermoplastic polyurethane, polyolefin elastomer, dynamically vulcanized polyolefin elastomer, polystyrene elastomer, polyether-ester elastomer, polyamide elastomer, and polyvinyl chloride.

According to one embodiment of the present disclosure, a reflective layer or a dichroic layer is disposed on an inner surface of the contact portion.

According to one embodiment of the present disclosure, the radial displacement pulse wave measuring device of the present disclosure further comprises a wearing portion configured to accommodate a limb portion of the subject where the measurement site is located in an internal space of the wearing portion. The wearing portion comprises a soft inner layer and a hard outer layer. The soft inner layer comprises the transparent airbag. The hard outer layer is disposed outside the soft inner layer to form the internal space of the wearing portion together with the soft inner layer. During a vascular radial displacement pulse wave measurement process, the hard outer layer maintains a distance between the displacement sensing module and a farthest position on the hard outer layer relative to the displacement sensing module as a fixed distance.

According to one embodiment of the present disclosure, the soft inner layer further comprises a plurality of auxiliary airbags, and an internal pressure of the auxiliary airbags is controlled by the pressure control module.

According to one embodiment of the present disclosure, the pressure control module comprises a pressure sensor and a pressure adjustment module. The pressure sensor is configured to sense an internal pressure of the transparent airbag. The pressure adjustment module is configured to adjust the internal pressure of the transparent airbag, and the pressure adjustment module comprises a pump.

According to one embodiment of the present disclosure, the pressure adjustment module further comprises a pulse width modulation circuit configured to adjust a rotational speed of a motor of the pump.

According to one embodiment of the present disclosure, the displacement sensing module comprises a transmitter and a receiver. The transmitter and the receiver are aligned with the transparent portion, and the transparent portion and the transparent airbag are configured such that a measurement signal emitted by the transmitter may pass through the transparent portion and the transparent airbag.

According to one embodiment of the present disclosure, the displacement sensing module comprises a photoelectric displacement sensor.

According to one embodiment of the present disclosure, the photoelectric displacement sensor comprises a distance measuring device selected from a group consisting of a laser displacement meter, a fiber-optic displacement sensor, a three-dimensional laser scanner, a time-of-flight (TOF) distance sensor, a three-dimensional time-of-flight (3D TOF) distance array sensor, a laser Doppler anemometer, a laser Doppler velocimeter, a laser Doppler vibrometer, a Michelson interferometer, or a laser interferometer.

According to one embodiment of the present disclosure, the displacement sensing module further comprises a filter.

According to one embodiment of the present disclosure, the radial displacement pulse wave measuring device further comprises a scanning position control module communicationally connected to the computing unit and configured to control the displacement sensing module to perform distance-measuring scan within the measurement site. The scanning position control module comprises a single-axis position controller or a dual-axis position controller.

Another aspect of the present disclosure is to provide a radial displacement pulse wave measuring device for measuring a vascular radial displacement pulse wave of an artery at a measurement site of a subject. The radial displacement pulse wave measuring device comprises a housing, a non-transparent airbag, a pressure control module, a displacement sensing module, and a computing unit.

The housing has a transparent portion, and the transparent portion comprises a hole or a first transparent plate. A non-transparent airbag is disposed below the transparent portion, and a main material of the non-transparent airbag is a material that is resistant to stretching deformation. The non-transparent airbag comprises a transparent window overlapping the transparent portion of the housing, and the transparent window comprises a transparent material that is resistant to stretching deformation or a second transparent plate. The pressure control module is configured to control an internal pressure of the non-transparent airbag to control a downward pressure applied by the non-transparent airbag to the measurement site to enhance a pulse signal of the artery. The displacement sensing module is disposed above the transparent portion. The displacement sensing module is configured to sense pulse beats of the artery and measure a dynamic distance between a skin of the measurement site and the displacement sensing module caused by the pulse beats of the artery to obtain the vascular radial displacement pulse wave. The computing unit is communicationally connected to the pressure control module and the displacement sensing module. The computing unit is configured to respectively transmit control signals to the pressure control module and the displacement sensing module, as well as respectively receive information transmitted from the pressure control module and the displacement sensing module for performing calculations.

According to one embodiment of the present disclosure, the transparent material that is resistant to stretching deformation of the main material of the non-transparent airbag comprises polymethyl methacrylate, cellulose acetate, nylon-66 polyamide resin, nylon-6 polyamide resin, polybutylene terephthalate, polyethylene terephthalate, polyphenylene oxide, polycarbonate, ethylene-vinyl acetate copolymer, polyethylene, polypropylene, polyvinyl chloride, polyoxymethylene, polyurethane, glass, quartz, polystyrene, or acrylonitrile-butadiene-styrene copolymer.

According to one embodiment of the present disclosure, the transparent material that is resistant to stretching deformation of the transparent window comprises polymethyl methacrylate, cellulose acetate, nylon-66 polyamide resin, nylon-6 polyamide resin, polybutylene terephthalate, polyethylene terephthalate, polyphenylene oxide, polycarbonate, ethylene-vinyl acetate copolymer, polyethylene, polypropylene, polyvinyl chloride, polyoxymethylene, polyurethane, glass, quartz, polystyrene, or acrylonitrile-butadiene-styrene copolymer.

According to one embodiment of the present disclosure, an anti-reflection coating is disposed on an outer surface of the transparent window.

According to one embodiment of the present disclosure, when the transparent portion is the hole, the transparent window is the second transparent plate.

According to one embodiment of the present disclosure, an area of the second transparent plate is larger than an area of the hole, such that when an internal pressure of the transparent airbag is excessive, the second transparent plate abuts against a lower side of the hole and is prevented from being pushed to an upper side of the hole.

According to one embodiment of the present disclosure, the non-transparent airbag further comprises a contact portion. The contact portion is configured to closely fit the skin of the measurement site of the subject, and a material of the contact portion is a soft, stretchable, and deformable thermoplastic elastomer material. The elastomer material comprises at least one of thermoplastic polyurethane, polyolefin elastomer, dynamically vulcanized polyolefin elastomer, polystyrene elastomer, polyether-ester elastomer, polyamide elastomer, and polyvinyl chloride.

According to one embodiment of the present disclosure, a reflective layer or a dichroic layer is disposed on an inner surface of the contact portion.

According to one embodiment of the present disclosure, the radial displacement pulse wave measuring device of the present disclosure further comprises a wearing portion configured to accommodate a limb portion of the subject where the measurement site is located in an internal space of the wearing portion. The wearing portion comprises a soft inner layer and a hard outer layer. The soft inner layer comprises the transparent airbag. The hard outer layer is disposed outside the soft inner layer to form the internal space of the wearing portion together with the soft inner layer. During a vascular radial displacement pulse wave measurement process, the hard outer layer maintains a distance between the displacement sensing module and a farthest position on the hard outer layer relative to the displacement sensing module as a fixed distance.

According to one embodiment of the present disclosure, the soft inner layer further comprises a plurality of auxiliary airbags, and an internal pressure of the auxiliary airbags is controlled by the pressure control module.

According to one embodiment of the present disclosure, the pressure control module comprises a pressure sensor and a pressure adjustment module. The pressure sensor is configured to sense an internal pressure of the transparent airbag. The pressure adjustment module is configured to adjust the internal pressure of the transparent airbag, and the pressure adjustment module comprises a pump.

According to one embodiment of the present disclosure, the pressure adjustment module further comprises a pulse width modulation circuit configured to adjust a rotational speed of a motor of the pump.

According to one embodiment of the present disclosure, the displacement sensing module comprises a transmitter and a receiver. The transmitter and the receiver are aligned with the transparent portion, and the transparent portion and the transparent window are configured such that a measurement signal emitted by the transmitter passes through the transparent portion and the transparent window.

According to one embodiment of the present disclosure, the displacement sensing module comprises a photoelectric displacement sensor.

According to one embodiment of the present disclosure, the photoelectric displacement sensor comprises a distance measuring device selected from a group consisting of a laser displacement meter, a fiber-optic displacement sensor, a three-dimensional laser scanner, a time-of-flight (TOF) distance sensor, a three-dimensional time-of-flight (3D TOF) distance array sensor, a laser Doppler anemometer, a laser Doppler velocimeter, a laser Doppler vibrometer, a Michelson interferometer, or a laser interferometer.

According to one embodiment of the present disclosure, the displacement sensing module further comprises a filter.

According to one embodiment of the present disclosure, the radial displacement pulse wave measuring device of the present disclosure further comprises a scanning position control module communicationally connected to the computing unit and configured to control the displacement sensing module to perform distance-measuring scan within the measurement site. The scanning position control module comprises a single-axis position controller or a dual-axis position controller.

Another aspect of the present disclosure is to provide a measurement condition optimization method for vascular radial displacement pulse wave. In this method, a Y-axis is defined as substantially perpendicular to a direction of the artery and substantially parallel to a surface of the skin at the measurement site. The method comprises: using the above-described radial displacement pulse wave measuring device; placing the transparent airbag or the non-transparent airbag on the skin of the measurement site of the subject; positioning the displacement sensing module at an initial position, where the displacement sensing module is a point-type displacement sensor or a matrix-type displacement sensor; maintaining a pressure of the transparent airbag or the non-transparent airbag at a first pressure, and pressing the transparent airbag or the non-transparent airbag against the measurement site to a first depth; scanning the displacement sensing module along the Y-axis on the surface of the measurement site until a first measurement position is found where a maximum amplitude signal of the vascular radial displacement pulse wave of the artery is obtained; keeping the displacement sensing module at the first measurement position and adjusting the pressure of the transparent airbag or the non-transparent airbag to find a second pressure at which a maximum signal of the vascular radial displacement pulse wave of the artery is obtained; and maintaining the pressure of the transparent airbag or the non-transparent airbag at the second pressure to press the transparent airbag or the non-transparent airbag against the measurement site to a second depth. The first measurement position and the second depth serve as measurement conditions for the vascular radial displacement pulse wave of the artery.

According to one embodiment of the present disclosure, the first pressure is obtained by gradually increasing the pressure of the transparent airbag or the non-transparent airbag to vertically press the measurement site with the transparent airbag or the non-transparent airbag until the first pressure is applied when a maximum amplitude signal of a vascular volume pulse wave of the artery is found.

According to one embodiment of the present disclosure, the measurement condition optimization method for vascular radial displacement pulse wave of the present disclosure further comprises scanning the displacement sensing module along the direction of the artery until a second measurement position is found where a local maximum signal of the vascular radial displacement pulse wave of the artery is obtained. The second measurement position replaces the first measurement position as the measurement condition for the vascular radial displacement pulse wave of the artery.

Another aspect of the present disclosure is to provide a measurement condition optimization method for vascular radial displacement pulse wave. The method comprises: finding a first measurement position above an artery at a measurement site of a subject; using the above-described radial displacement pulse wave measuring device; placing the transparent airbag or the non-transparent airbag on the skin of the measurement site of the subject; aligning the displacement sensing module with the first measurement position, where the displacement sensing module is a point-type displacement sensor or a matrix-type displacement sensor; gradually increasing the pressure of the transparent airbag or the non-transparent airbag to vertically press the measurement site with the transparent airbag or the non-transparent airbag until a measurement pressure is applied when a maximum signal of the vascular radial displacement pulse wave of the artery is found; maintaining the pressure of the transparent airbag or the non-transparent airbag at the measurement pressure and pressing the transparent airbag or the non-transparent airbag against the measurement site to a measurement depth. The first measurement position and the measurement depth serve as measurement conditions for the vascular radial displacement pulse wave of the artery.

According to one embodiment of the present disclosure, the measurement condition optimization method for vascular radial displacement pulse wave of the present disclosure further comprises scanning the displacement sensing module along the direction of the artery until a second measurement position is found where a local maximum signal of the vascular radial displacement pulse wave of the artery is obtained. The second measurement position replaces the first measurement position as the measurement condition for the vascular radial displacement pulse wave of the artery.

Another aspect of the present disclosure is to provide a measurement condition optimization method for vascular radial displacement pulse wave. The method comprises: using the above-described radial displacement pulse wave measuring device; placing the transparent airbag or the non-transparent airbag on the skin of the measurement site of the subject; maintaining a pressure of the transparent airbag or the non-transparent airbag at a first pressure; moving the displacement sensing module above the measurement site. The displacement sensing module is a matrix-type displacement sensor, and the matrix-type displacement sensor has a measurement region that intersects with a direction of the blood vessel. Among the point-type displacement sensors within the measurement region, one corresponding to a maximum amplitude signal is identified, indicating that this displacement sensor is located at a first measurement position. The pressure of the transparent airbag or the non-transparent airbag is then adjusted to find a second pressure applied when a maximum signal of the vascular radial displacement pulse wave of the artery is obtained. The pressure of the transparent airbag or the non-transparent airbag is maintained at the second pressure to press the transparent airbag or the non-transparent airbag against the measurement site to a second depth. The first measurement position and the second depth serve as measurement conditions for the vascular radial displacement pulse wave of the artery.

According to one embodiment of the present disclosure, the first pressure is obtained by gradually increasing the pressure of the transparent airbag or the non-transparent airbag to vertically press the measurement site with the transparent airbag or the non-transparent airbag until the first pressure is applied when a maximum amplitude signal of a vascular volume pulse wave of the artery is found.

According to one embodiment of the present disclosure, the measurement condition optimization method for vascular radial displacement pulse wave of the present disclosure further comprises scanning the displacement sensing module along the direction of the artery until a second measurement position is found where a local maximum signal of the vascular radial displacement pulse wave of the artery is obtained. The second measurement position replaces the first measurement position as the measurement condition for the vascular radial displacement pulse wave of the artery.

Another aspect of the present disclosure is to provide a blood pressure measurement method. The method comprises: using the above-described radial displacement pulse wave measuring device; placing the transparent airbag or the non-transparent airbag on the measurement site; using the above-described measurement condition optimization method for vascular radial displacement pulse wave to locate the artery at the measurement site of the subject for measuring the vascular radial displacement pulse wave of the artery; gradually pressurizing the transparent airbag or the non-transparent airbag until the vascular radial displacement pulse wave of the artery appears and then disappears; gradually depressurizing the transparent airbag or the non-transparent airbag until the vascular radial displacement pulse wave of the artery begins to appear, where the pressure of the transparent airbag or the non-transparent airbag at this moment is the systolic pressure of the artery; and continuously gradually depressurizing the transparent airbag or the non-transparent airbag until the vascular radial displacement pulse wave of the artery begins to disappear, where the pressure of the transparent airbag or the non-transparent airbag at this moment is the diastolic pressure of the artery.

Another aspect of the present disclosure is to provide a pulse wave velocity (PWV) measurement method. The method comprises: measuring a proximal radial displacement pulse wave at a proximal measurement position of a proximal artery portion of a subject, which is closer to the heart, by using the above-described radial displacement pulse wave measuring device while simultaneously recording an electrocardiogram (ECG); simultaneously recording the vascular radial displacement pulse wave of the proximal artery portion and the ECG of the subject to obtain a delay time T₁ of the proximal radial displacement pulse wave of the proximal artery portion relative to the R-wave of the ECG; measuring a distal radial displacement pulse wave at a distal measurement position of a distal artery portion of the subject, which is farther from the heart, by using the above-described radial displacement pulse wave measuring device while simultaneously recording an electrocardiogram (ECG); simultaneously recording the vascular radial displacement pulse wave of the distal artery portion and the ECG of the subject to obtain a delay time T₂ of the distal radial displacement pulse wave of the distal artery portion relative to the R-wave of the ECG; calculating a delay time difference Δ=T₂−T₁; measuring a distance ΔD between the proximal artery portion and the distal artery portion; and calculating the pulse wave velocity PWV=ΔD/ΔT.

According to one embodiment of the present disclosure, the proximal measurement position and the distal measurement position are the first measurement position found by using the above-described measurement condition optimization method for vascular radial displacement pulse wave, or the second measurement position found by using the above-described measurement condition optimization method for vascular radial displacement pulse wave.

Another aspect of the present disclosure is to provide a pulse wave velocity (PWV) measurement method. The method comprises: measuring a proximal radial displacement pulse wave at a proximal measurement position of a proximal artery portion of a subject, which is closer to the heart, without recording an electrocardiogram (ECG); simultaneously measuring a distal radial displacement pulse wave at a distal measurement position of a distal artery portion of the subject, which is farther from the heart, without recording an electrocardiogram (ECG), where the proximal measurement position and the distal measurement position use one or two of the above-described radial displacement pulse wave measuring devices to simultaneously measure the proximal radial displacement pulse wave and the distal radial displacement pulse wave; calculating a delay time difference ΔT between the proximal radial displacement pulse wave and the distal radial displacement pulse wave; measuring a distance ΔD between the proximal artery portion and the distal artery portion; and calculating the pulse wave velocity PWV=ΔD/ΔT.

According to one embodiment of the present disclosure, the proximal measurement position and the distal measurement position are the first measurement position found by using the above-described measurement condition optimization method for vascular radial displacement pulse wave, or the second measurement position found by using the above-described measurement condition optimization method for vascular radial displacement pulse wave.

Another aspect of the present disclosure is to provide a method for continuously measuring blood pressure. The method comprises: using the above-described blood pressure measurement method to measure a diastolic pressure of a subject under an initial state and a corresponding first pressing depth of the transparent airbag or the non-transparent airbag on the measurement site, and to measure a systolic pressure of the subject under the initial state and a corresponding second pressing depth of the transparent airbag or the non-transparent airbag on the measurement site, where a difference between the first pressing depth and the second pressing depth is a vascular diameter R of the subject; calculating a vascular diameter variation ΔR of the subject from a time-dependent waveform of the radial displacement pulse wave of the subject; using the above-described pulse wave velocity measurement method to measure a pulse wave velocity PWV of the subject; obtaining a blood density ρ of the subject; calculating a blood pressure variation ΔP using the following Bramwell-Hill formula: ΔP=ρ×PWV²(ΔR/R); and calculating a real-time systolic pressure (=initial systolic pressure+ΔP) and a real-time diastolic pressure (=initial diastolic pressure+ΔP) of the subject.

Still another aspect of the present disclosure is to provide a radial displacement pulse wave measuring device for measuring a vascular radial displacement pulse wave of an artery at a measurement site of a subject. The device comprises a housing, an airbag, a pressure control module, a displacement sensing module, and a computing unit. The housing has a transparent portion. The airbag is disposed beneath the transparent portion of the housing and comprises a transparent window and a contact portion. The transparent window overlaps the transparent portion of the housing, and the contact portion is configured to be in close contact with the skin of the measurement site. The pressure control module is pneumatically connected to the airbag via a gas pipeline. The pressure control module comprises a pressure sensor configured to sense an internal pressure of the airbag, and a pressure adjustment module configured to adjust the internal pressure of the airbag. The pressure sensor is further configured to measure a vascular volumetric pulse wave of the artery. The displacement sensing module is disposed above the transparent portion and is configured to emit and receive a measurement signal that penetrates the transparent portion of the housing and the transparent window of the airbag. By measuring a dynamic distance between the displacement sensing module and the measurement site based on the signal variations caused by arterial pulse beats, the displacement sensing module is thereby configured to measure a vascular radial displacement pulse wave of the artery.

The computing unit is communicatively connected to the pressure control module and the displacement sensing module and is configured to execute a series of operations to optimize the measurement of vascular radial displacement pulse waves. First, the computing unit gradually increases the internal pressure of the airbag via the pressure adjustment module. When the pressure sensor detects that the vascular volumetric pulse wave signal reaches its peak, the computing unit determines this pressure as the first pressure value. Next, the computing unit maintains the internal pressure of the airbag at the first pressure value via the pressure adjustment module. Subsequently, the displacement sensing module and the measurement site move relative to each other in a direction substantially perpendicular to the orientation of the artery, either manually or automatically. During this relative movement, a first distance measuring scan is performed. Based on the scan results, the computing unit identifies the position where the vascular radial displacement pulse wave signal reaches its maximum. This position is designated as the first measurement position, which corresponds to the actual location of the artery. The computing unit then further adjusts the internal pressure of the airbag based on the identified measurement position via the pressure adjustment module. When the radial displacement pulse wave signal again reaches a peak under the adjusted condition, the computing unit defines this pressure as the second pressure value. Finally, the computing unit maintains the airbag at the second pressure value to apply a stable pressing depth onto the first measurement position. Under this condition, the displacement sensing module can accurately measure the vascular radial displacement pulse wave. The first measurement position, the second pressure value and the pressing depth constitute a first set of measurement conditions for measuring the vascular radial displacement pulse wave of the artery.

According to one embodiment of the present disclosure, while the internal pressure of the airbag is maintained at the second pressure value, the displacement sensing module and the measurement site move relative to each other along the orientation of the artery, either manually or automatically. During this relative movement, the computing unit performs a second distance measuring scan via the displacement sensing module and identifies a second measurement position at which a local maximum of the vascular radial displacement pulse wave signal is detected. The first measurement position is replaced by the second measurement position, thereby establishing a second set of measurement conditions for measuring the vascular radial displacement pulse wave of the artery.

Still another aspect of the present disclosure is to provide a radial displacement pulse wave measuring device for measuring a vascular radial displacement pulse wave of an artery at a measurement site of a subject. The device comprises a housing, an airbag, a pressure control module, a displacement sensing module, a computing unit, and a wearing portion. The housing has a transparent portion. The airbag is disposed beneath the transparent portion of the housing and comprises a transparent window and a contact portion. The transparent window overlaps the transparent portion, and the contact portion is configured to be in close contact with the skin of the measurement site. The pressure control module is pneumatically connected to the airbag via a gas pipeline. The pressure control module includes a pressure sensor and a pressure adjustment module. The pressure sensor is configured to sense an internal pressure of the airbag and to measure a vascular volumetric pulse wave of the artery. The pressure adjustment module is configured to adjust the internal pressure of the airbag. The displacement sensing module is disposed above the transparent portion and is configured to emit and receive a measurement signal that penetrates both the transparent portion of the housing and the transparent window of the airbag. Through this configuration, the displacement sensing module measures dynamic changes in distance between itself and the measurement site, thereby obtaining a vascular radial displacement pulse wave. The computing unit is communicatively connected to the pressure control module and the displacement sensing module. The computing unit is configured to transmit control signals to both the pressure control module and the displacement sensing module, and to receive information therefrom for performing calculations. The wearing portion is disposed below the housing and is configured to stably accommodate a limb portion of the subject at the measurement site within an internal space of the wearing portion. The wearing portion includes a soft inner layer and a hard outer layer. The soft inner layer comprises the airbag. The hard outer layer is disposed outside the soft inner layer and cooperates therewith to form the internal space. During measurement of the vascular radial displacement pulse wave, the hard outer layer is configured such that the distance between the displacement sensing module and the position on the hard outer layer farthest from the displacement sensing module remains fixed.

Still another aspect of the present disclosure is to provide a radial displacement pulse wave measuring device for measuring a vascular radial displacement pulse wave of an artery at a measurement site of a subject. The device comprises an airbag, a pressure control module, a displacement sensing module, and a computing unit. The airbag comprises a transparent window and a contact portion. The contact portion is configured to be in close contact with the skin of the measurement site. The pressure control module is pneumatically connected to the airbag via a gas pipeline. The pressure control module includes a pressure sensor configured to sense an internal pressure of the airbag, and a pressure adjustment module configured to adjust the internal pressure of the airbag. The displacement sensing module is configured to emit and receive a measurement signal that penetrates the transparent window of the airbag to measure a dynamic distance between the displacement sensing module and the measurement site.

The computing unit is communicatively connected to the pressure control module and the displacement sensing module, and is configured to execute a series of operations to facilitate the measurement of vascular radial displacement pulse waves. First, the computing unit gradually increases the internal pressure of the airbag via the pressure adjustment module. When the displacement sensing module detects that the vascular radial displacement pulse wave signal reaches a maximum, the computing unit identifies this pressure as the first pressure value. Next, the computing unit maintains the internal pressure of the airbag at the first pressure value via the pressure adjustment module. Subsequently, the displacement sensing module and the measurement site move relative to each other in a direction substantially perpendicular to the orientation of the artery, either manually or automatically. During this relative movement, a first distance measuring scan is performed. Based on the scan results, the computing unit identifies the position where the vascular radial displacement pulse wave signal reaches its peak. This position is designated as the first measurement position, which corresponds to the actual location of the artery. The computing unit then adjusts the internal pressure of the airbag based on the identified measurement position via the pressure adjustment module. When the radial displacement pulse wave signal again reaches a peak under the adjusted condition, the computing unit defines this pressure as the second pressure value. Finally, the computing unit maintains the airbag at the second pressure value to ensure a stable pressing depth at the first measurement position, thereby enabling accurate measurement of the vascular radial displacement pulse wave by the displacement sensing module. The first measurement position, the second pressure value and the pressing depth constitute a first set of measurement conditions for measuring the vascular radial displacement pulse wave of the artery.

According to one embodiment of the present disclosure, while the internal pressure of the airbag is maintained at the second pressure value, the displacement sensing module and the measurement site move relative to each other along the orientation of the artery, either manually or automatically. During this relative movement, the computing unit performs a second distance measuring scan via the displacement sensing module and identifies a second measurement position at which a local maximum of the vascular radial displacement pulse wave signal is detected. Then, the first measurement position is replaced with the second measurement position, thereby establishing a second set of measurement conditions for measuring the vascular radial displacement pulse wave of the artery.

The above-described radial displacement pulse wave measuring device has the following technical advantages. First, the radial displacement pulse wave measuring device can directly measure vascular radial displacement pulse waves, providing more accurate results compared to conventional pressure pulse wave measurement methods. Second, the radial displacement pulse wave measuring device has high precision, with a resolution of less than 100 micrometers, providing detailed pulse wave information, which is beneficial for various pulse wave analysis applications. Third, the radial displacement pulse wave measuring device can be applied to many non-invasive measurement methods, offering convenience and stability, real-time measurement results, and waveform displays, while also allowing long-term continuous monitoring. With these advantages, the radial displacement pulse wave measuring device has broad application value in the fields of cardiovascular medicine and biomedical science, supporting improved personalized healthcare and health monitoring.

### BRIEF DESCRIPTION OF THE DRAWINGS

FIG. 1 is a schematic functional block diagram of a radial displacement pulse wave measuring device according to one embodiment of the present disclosure.

FIG. 2 is a schematic cross-sectional diagram of a wearing portion of a radial displacement pulse wave measuring device according to one embodiment of the present disclosure.

FIG. 3A is a schematic diagram illustrating an operation flow for automatically locating a measurement point of a radial displacement pulse wave by using a scanning position control module 140 according to one embodiment of the present disclosure, where the displacement sensing module 130 is a point-type displacement sensor.

FIG. 3B is a schematic diagram illustrating an operation flow for manually locating a measurement point of a radial displacement pulse wave according to another embodiment of the present disclosure, where the displacement sensing module 130 is a point-type displacement sensor.

FIG. 3C is a schematic diagram illustrating an operation flow for locating a measurement point of a radial displacement pulse wave according to yet another embodiment of the present disclosure, where the displacement sensing module 130 is a linear matrix-type displacement sensor.

FIG. 4 is a schematic diagram illustrating waveform changes of a vascular volume pulse wave and a radial displacement pulse wave with respect to pressure and time according to one embodiment of the present disclosure.

FIG. 5 is a schematic diagram illustrating a process of measuring blood pressure by using the radial displacement pulse wave measuring device 100 shown in FIG. 1.

FIG. 6A is a schematic enlarged view of a radial displacement pulse wave during period III (t2-t3) in FIG. 4.

FIG. 6B is a diagram illustrating a variation of beat-to-beat intervals over time.

FIG. 6C is a spectral distribution diagram of RRI obtained by performing discrete Fourier transform (DFT) on heart rate variability (HRV).

FIG. 7A is a schematic diagram of simultaneously using two pulse wave measuring devices to measure pulse wave velocity according to one embodiment of the present disclosure.

FIG. 7B is a schematic diagram of measuring pulse wave velocity with ECG assistance according to one embodiment of the present disclosure.

FIG. 7C is a flowchart of a pulse wave velocity measurement method using the approach shown in FIG. 7A.

FIG. 8 is a flowchart of a method for continuously measuring blood pressure.

### DETAILED DESCRIPTION

Accordingly, the present disclosure provides a radial displacement pulse wave measuring device and an application method thereof. The radial displacement pulse wave measuring device can directly measure a waveform of a vascular radial displacement pulse wave through a coordinated design of the airbag, the pressure control module, and the displacement sensing module, with a measurement accuracy of less than 100 micrometers. Therefore, the device can provide many details of the waveform of the vascular radial displacement pulse wave for various application analyses of the radial displacement pulse wave. The following describes the measurement of blood pressure, heart rate variability, pulse wave velocity, and continuous measurement of blood pressure. These measurement methods are merely a few examples of the applications of the radial displacement pulse wave measuring device, and the application methods of the radial displacement pulse wave measuring device are not limited thereto.

Definition of coordinate axis directions: The XY plane is substantially parallel to a skin surface of a subject, where the X-axis is substantially parallel to a direction of a blood vessel of the subject, and the Y-axis is substantially perpendicular to the direction of the blood vessel. Therefore, the Z-axis is substantially perpendicular to the skin surface of the subject. The definitions of the X-axis, Y-axis, and Z-axis mentioned in the following descriptions are the same as defined above.

### Radial Displacement Pulse Wave Measuring Device

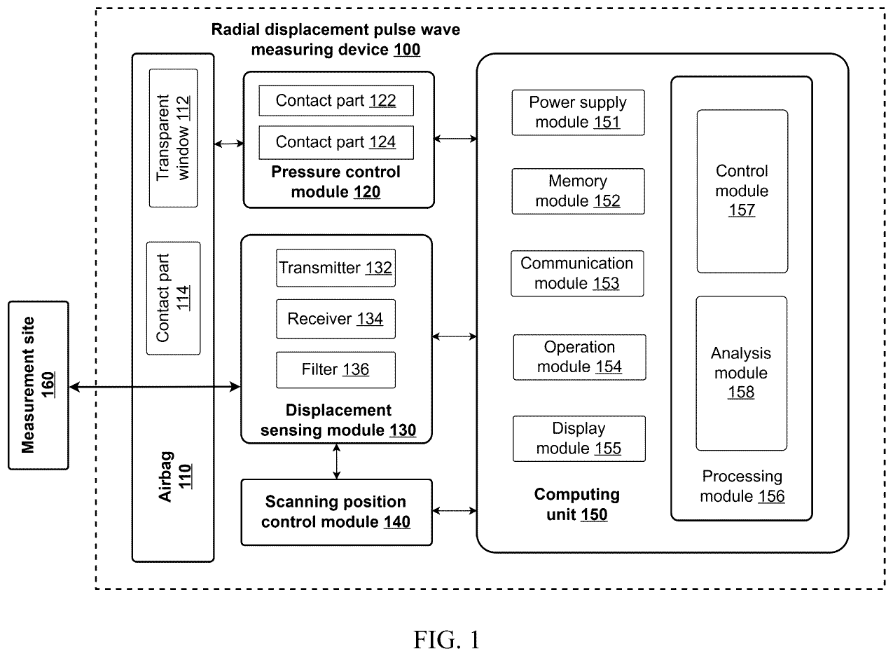

FIG. 1 is a schematic functional block diagram of a radial displacement pulse wave measuring device according to one embodiment of the present disclosure. As shown in FIG. 1, a radial displacement pulse wave measuring device 100 comprises an airbag 110, a pressure control module 120, a displacement sensing module 130, a scanning position control module 140, and a computing unit 150. The pressure control module 120, the displacement sensing module 130, and the scanning position control module 140 are communicationally connected to the computing unit 150, respectively.

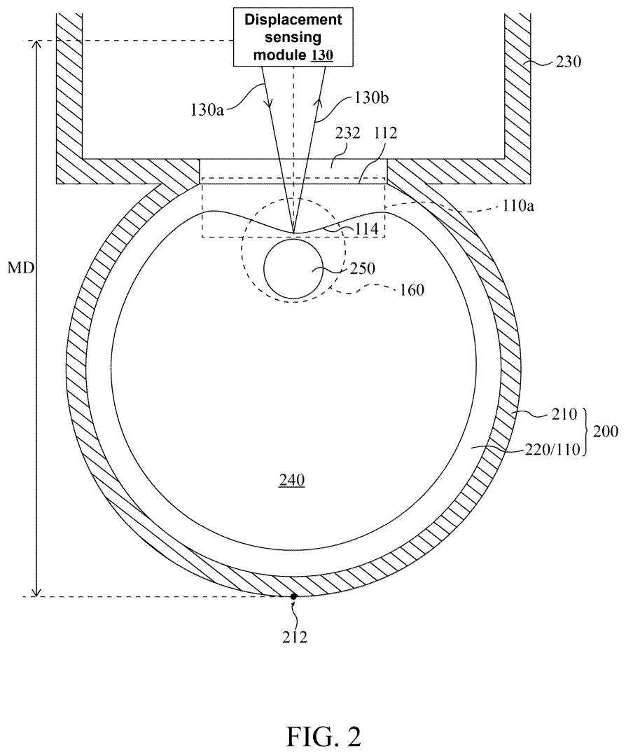

FIG. 2 is a schematic cross-sectional diagram of a wearing portion of a radial displacement pulse wave measuring device according to one embodiment of the present disclosure. To allow the airbag 110 and a measurement site 160 of a subject to stably and closely fit together, to fix a relative position between the displacement sensing module 130 and the measurement site 160 of the subject, and to effectively reduce noise caused by posture changes or body tremors of the subject, a wearing portion is designed for the radial displacement pulse wave measuring device 100 to improve the accuracy of pulse wave measurement. As shown in FIG. 2, the radial displacement pulse wave measuring device 100 further comprises a wearing portion 200 and a housing 230. The wearing portion 200 comprises a hard outer layer 210 and a soft inner layer 220, and the housing 230 has a transparent portion 232. In the radial displacement pulse wave measuring device 100, the airbag 110 is disposed below the transparent portion 232 (outside the housing 230), and the displacement sensing module 130 is disposed above the transparent portion 232 (inside the housing 230). The computing unit 150, the pressure control module 120, and the scanning position control module 140 may be optionally enclosed inside the housing 230 or disposed outside the housing 230. To simplify the complexity of the drawing, only the displacement sensing module 130 is shown inside the housing 230 in FIG. 2. When the wearing portion 200 and the housing 230 shown in FIG. 2 are used, the airbag 110 is located between the measurement site 160 of the subject and the housing 230, and the displacement sensing module 130 is aligned with the transparent portion 232 of the housing 230, so that an emitted signal 130a and a reflected signal 130b of the displacement sensing module 130 can pass through the transparent portion 232.

The following describes the components of the radial displacement pulse wave measuring device 100 one by one.

As shown in FIGS. 1 and 2, the airbag 110 has three functions. The first function is to generate a downward pressure to compress subcutaneous tissue. The second function is to allow a measurement signal of the displacement sensing module 130 (i.e., the emitted signal 130a and the reflected signal 130b) to pass through an internal space of the airbag 110 and be reflected back to the displacement sensing module 130. The third function is that when the airbag 110 is tightly attached to the skin, the airbag 110 can serve as a space to accommodate the expansion of the diameter of the artery during each pulse beat.

A main material of the airbag 110 may be made of any available polymer material that is resistant to stretching deformation and is transparent, or may be made of any available polymer material that is resistant to stretching deformation and is non-transparent. In the present disclosure, the term “non-transparent” includes opaque and semi-transparent. The transparent or non-transparent polymer material may be, but is not limited to, polymethyl methacrylate (PMMA), cellulose acetate (CA), nylon-66 polyamide resin (PA-66), nylon-6 polyamide resin (PA-6), polybutylene terephthalate (PBT), polyethylene terephthalate (PET), polyphenylene oxide (PPO), polycarbonate (PC), ethylene-vinyl acetate copolymer (EVA), polyethylene (PE), polypropylene (PP), polyvinyl chloride (PVC), polyoxymethylene (POM), or polyurethane (PU).

When the airbag 110 is made of a transparent polymer material, a measurement signal of the displacement sensing module 130 (i.e., the emitted signal 130a and the reflected signal 130b) can pass through a main material of the airbag 110. When a transparent portion 232 of the housing 230 shown in FIG. 2 is a hole, in order to increase the transmittance of the measurement signal of the displacement sensing module 130 through the main material of the airbag 110, or to reduce refraction and reflection of the measurement signal of the displacement sensing module 130 by the main material of the airbag 110, which may affect measurement accuracy, an upper portion of the airbag 110 made of the transparent polymer material may further comprise a transparent window 112 made of a transparent plate that is resistant to deformation. An area of the transparent plate of the transparent window 112 is larger than an area of the hole of the transparent portion 232 to prevent the transparent plate of the transparent window 112 from being squeezed into an interior of the housing 230 when a pressure of the airbag 110 is too high. During use, the transparent window 112 of the airbag 110 needs to be aligned with the transparent portion 232 of the housing 230 (i.e., the transparent window 112 and the transparent portion 232 at least partially overlap) to allow the measurement signal of the displacement sensing module 130 (i.e., the emitted signal 130a and the reflected signal 130b) to vertically pass through the transparent window 112 made of the transparent plate and enter an internal space of the airbag 110. When the transparent portion 232 of the housing 230 shown in FIG. 2 is a transparent plate that is resistant to deformation, the airbag 110 made of the transparent polymer material does not require a transparent window 112 made of a transparent plate. In addition, regardless of whether the transparent portion 232 of the housing 230 is a hole or a transparent plate, since the housing 230 is located above the airbag 110, the housing 230 can also serve as a limiting baffle to restrict upward expansion of the airbag 110 during pressurization. As a result, the transparent window 112 that contacts the transparent portion 232 is maintained in a flat state, and the increased pressure in the airbag 110 is almost entirely used to press downward against the measurement site 160 of the subject.

However, when the airbag 110 is made of a non-transparent polymer material, a measurement signal of the displacement sensing module 130 cannot easily pass through, or can only partially pass through, a main material of the airbag 110. Therefore, the airbag 110 needs to at least have a transparent window 112 to allow the measurement signal of the displacement sensing module 130 to pass through. During use, the transparent window 112 of the airbag 110 also needs to be aligned with the transparent portion 232 of the housing 230 (i.e., the transparent window 112 and the transparent portion 232 at least partially overlap) to allow the measurement signal of the displacement sensing module 130 (i.e., the emitted signal 130a and the reflected signal 130b) to pass through the transparent window 112 and the transparent portion 232 and enter an internal space of the airbag 110. When the transparent portion 232 of the housing 230 shown in FIG. 2 is a hole, the transparent window 112 of the airbag 110 made of the non-transparent polymer material needs to be made of a transparent plate that is resistant to deformation, and an area of the transparent plate of the transparent window 112 is larger than an area of the hole of the transparent portion 232 to prevent the transparent plate of the transparent window 112 from being squeezed into an interior of the housing 230 when an internal pressure of the airbag 110 is too high. The remaining aspects are similar to the situation where the airbag 110 is made of the transparent polymer material, and thus, details will not be repeated. When the transparent portion 232 of the housing 230 shown in FIG. 2 is a transparent plate that is resistant to deformation, the transparent material used for the transparent window 112 of the airbag 110 made of the non-transparent polymer material is not limited to the transparent plate and may also be a transparent material that is resistant to stretching deformation. The transparent material that is resistant to stretching deformation may be, for example, but is not limited to, polymethyl methacrylate, cellulose acetate, nylon-66 polyamide resin, nylon-6 polyamide resin, polybutylene terephthalate, polyethylene terephthalate, polyphenylene oxide, polycarbonate, ethylene-vinyl acetate copolymer, polyethylene, polypropylene, polyvinyl chloride, polyoxymethylene, or polyurethane. The remaining aspects are similar to the situation where the airbag 110 is made of the transparent polymer material, and thus, the details will not be repeated.

Therefore, in FIG. 2, regardless of whether the transparent window 112 of the airbag 110 is formed by a transparent plate or the transparent portion 232 of the housing 230, both can restrict upward expansion of the airbag 110, so that the increased pressure in the airbag 110 is almost entirely used to press downward against the measurement site 160 of the subject through a contact portion 114 of the airbag 110. As a result, vascular radial displacement generated by pulse beats can be maximized, facilitating measurement by the displacement sensing module 130.

The transparent plate that is resistant to deformation and forms the transparent window 112 or the transparent portion 232 may be made of, but is not limited to, glass, quartz, polystyrene (PS), acrylonitrile butadiene styrene (ABS), or the same transparent material used for the airbag. The transparent window 112 and the transparent portion 232 may be made of the same material or different materials. According to some embodiments, the transparent portion 232 of the housing 230 and the transparent window 112 of the airbag 110 may also be integrated into a single structure, as long as a single transparent plate with high hardness is used.

According to further embodiments, an anti-reflection coating may be applied to an outer surface of the transparent window 112 facing an outer side of the airbag 110 to increase the transmittance of the measurement signal of the displacement sensing module 130 through the transparent window 112, thereby reducing noise caused by partial reflection of the measurement signal from the transparent window 112. The anti-reflection coating can also improve the wear resistance of the transparent window 112.

Since the main material of the airbag 110 is made of a material that is resistant to stretching deformation, it may reduce the amplitude of the reflection surface of the skin or the airbag at the area where the airbag tightly contacts the skin. In order to reduce lateral tension of the material while maintaining downward pressure inside the airbag, a soft polymer material with stretchable and deformable properties may be locally used. The airbag 110 may further comprise a contact portion 114, as shown in FIGS. 1 and 2. The contact portion 114 is used to contact a skin surface of the measurement site 160 of the subject, and the skin of the measurement site 160 may move up and down due to pulse beats of an artery 250 located beneath the skin (see FIG. 2). Therefore, the contact portion 114 can be made of a soft polymer material (with a hardness ranging from Shore 20C to 72D) that is stretchable and deformable to help reduce lateral tension exerted on the constantly moving skin of the measurement site 160. In cooperation with the main body of the airbag 110 and the transparent window 112 thereof, both of which are resistant to deformation, the soft and elastic contact portion 114 can effectively transmit the downward pressure of the airbag 110 onto the measurement site 160. Alternatively, the airbag 110 may be entirely made of the same material as the airbag that is resistant to stretching deformation, and after inflation, an elastic air layer with a compressible volume inside the airbag 110 may serve as an elastic buffer space required for pulse beats, while reducing lateral tension of the material, so that the displacement sensing module 130 can more easily detect a measurement signal corresponding to vascular radial displacement caused by pulse beats. The contact portion 114 may be transparent, semi-transparent, or non-transparent. The above-mentioned polymer material may be the same as the main material of the airbag or may be, for example, a thermoplastic elastomer (TPE). The available thermoplastic elastomer may be, for example, thermoplastic polyurethane (TPU), polyolefin elastomer (TPO), dynamically vulcanized polyolefin elastomer (TPV), polystyrene-based elastomer (TPS/TPR), polyether-ester elastomer (TPEE), polyamide-based elastomer (TPA), or polyvinyl chloride (PVC).

According to some embodiments, an inner surface of the contact portion 114 facing the airbag 110 may be, for example, smooth, or a reflective layer or a dichroic layer may additionally be formed on the inner surface of the contact portion 114. For example, when a red laser light source is used, a dichroic layer coated on the inner surface of the contact portion 114 of the airbag 110 can reflect red laser light while allowing other visible light to pass through, thereby increasing the reflection intensity of the measurement signal (i.e., the red laser) and facilitating alignment with an alignment mark located below the contact portion 114. In this way, the reflection intensity and uniformity (rather than scattering) of the measurement signal emitted by the displacement sensing module 130 on the inner surface of the contact portion 114 can be increased. The contact portion 114 may also be semi-transparent to simultaneously enhance the reflection intensity of the measurement signal and facilitate alignment with an alignment mark or reflective sticker located below the contact portion 114.

The pressure control module 120 shown in FIG. 1 controls an internal pressure of the airbag 110 by inflating and pressurizing or deflating and depressurizing the airbag 110 to adjust a downward pressing depth of the airbag 110 onto the measurement site 160. According to some embodiments, the pressure control module 120 may comprise a pressure sensor 122 and a pressure adjustment module 124 (including a pump, an air pipeline, and an air valve) that are communicationally connected to each other. The pressure sensor 122 is configured to detect the internal pressure of the airbag 110 and thus can be used to measure a vascular volume pulse wave of the subject. Two ends of the air pipeline of the pressure adjustment module 124 are respectively connected to the pump and the airbag 110, and the air valve is installed at a suitable position in the air pipeline. Therefore, the amount of gas entering and exiting the airbag 110 can be rapidly and precisely controlled by controlling the rotational direction (forward or reverse) of a motor in the pump, by using a pulse width modulation (PWM) circuit to adjust a rotational speed of the motor in the pump, by using two motors in the pump that are respectively responsible for air intake and exhaust, or in combination with switching of the air valve. As a result, the internal pressure of the airbag 110 can be rapidly and precisely controlled, thereby controlling a downward pressing depth of the airbag 110 along a Z-axis on a skin surface of the measurement site 160 of the subject. Accordingly, the above-mentioned pump, in combination with the air valve, the PWM circuit, or both, together with detection of a maximum amplitude of the vascular volume pulse wave, enables rapid determination of the required airbag pressure, thereby rapidly determining an approximate pressing depth of the airbag 110 on the skin of the subject that is suitable for detecting a vascular radial displacement pulse wave.

The displacement sensing module 130 shown in FIGS. 1 and 2 is configured to measure a distance in a Z-axis direction from the displacement sensing module 130 to a skin surface of the measurement site 160 of the subject (when the contact portion 114 of the airbag 110 is made of a transparent material), or to measure a distance in the Z-axis direction from the displacement sensing module 130 to the contact portion 114 of the airbag 110 (when the contact portion 114 of the airbag 110 is made of a non-transparent material). As a result, the displacement sensing module 130 can measure a vascular radial displacement pulse wave of the subject, which is hereinafter simply referred to as a radial displacement pulse wave.

The displacement sensing module 130 may be any available displacement sensor, which comprises a transmitter 132 for emitting a measurement signal and a receiver 134 for receiving the reflected measurement signal. The displacement sensing module 130 may be classified based on a shape of a maximum detectable area of the displacement sensing module 130. The displacement sensing module 130 may be a point-type displacement sensor or a matrix-type displacement sensor formed by arranging multiple point-type displacement sensors, such as a linear or planar matrix-type displacement sensor. A measurement resolution of the displacement sensing module 130 may be less than 100 micrometers, for example, the displacement sensing module 130 may have a resolution of 100 micrometers, 90 micrometers, 80 micrometers, 70 micrometers, 60 micrometers, 50 micrometers, 40 micrometers, 30 micrometers, 20 micrometers, 10 micrometers, 1 micrometer, or less than 1 micrometer.

The displacement sensor may be, for example, a photoelectric displacement sensor that performs distance measurement using light sources (i.e., the above-mentioned transmitter 132) of various wavelengths. The photoelectric displacement sensor may be, for example, a laser displacement meter, a fiber-optic displacement sensor, a three-dimensional laser scanner (3D laser scanner), such as a binocular depth CCD combined with a programmable structured light system, a time-of-flight (TOF) distance sensor, a three-dimensional time-of-flight (3D TOF) distance array sensor, a laser Doppler anemometer, a laser Doppler velocimeter, a laser Doppler vibrometer, a Michelson interferometer, or a laser interferometer.

According to some embodiments, the displacement sensing module 130 may further comprise a filter 136 configured to remove various noise mixed into the measurement signal, thereby further increasing a signal-to-noise ratio of the measurement signal.

The scanning position control module 140 shown in FIG. 1 is configured to control the displacement sensing module 130 to move above the measurement site 160 of the subject and to control the displacement sensing module 130 to perform distance-measuring scan within a range of the measurement site 160. By combining coordinates determined by position control and an amplitude of the radial displacement pulse wave measured at each specific location by the displacement sensing module 130, the location of a blood vessel in the measurement site 160 of the subject can be found, or a measurement location with a better signal-to-noise ratio along the direction of the blood vessel can be identified to obtain an optimal measurement signal of the radial displacement pulse wave. Therefore, the scanning position control module 140 is an optional module that may be omitted. When the scanning position control module 140 is not provided, a user may manually move the displacement sensing module 130 to the measurement site 160 of the subject to perform distance-measuring scan on the measurement site 160, or manually locate the position where the pulse beats most strongly and attach a reflective sticker, allowing a laser light source to be aligned with the reflective sticker. The displacement sensing module 130 may be a photoelectric displacement sensor having a detection area of a point type, a linear type, or a planar type. According to some embodiments, the scanning position control module 140 may comprise, for example, a single-axis position controller or a dual-axis position controller (such as an X-Y dual-axis moving platform or a cylindrical coordinate moving mechanism).

The computing unit 150 shown in FIG. 1 comprises multiple modules formed by a plurality of hardware circuits that are electrically connected to each other. The computing unit 150 may be any available machine with computing capability, such as various types of computers, microprocessors, cloud computing units, mobile computing devices, or edge computing devices for artificial intelligence. According to some embodiments, the computing unit 150 comprises a power module 151, a memory module 152, a communication module 153, an operation module 154, a display module 155, and a processing module 156, which are electrically or communicationally connected to each other. Some of these modules may also be integrated into a single chip.

The power module 151 is used to supply the electrical power required by the computing unit 150. The power module 151 may be an alternating current (AC) power source (for example, power supplied by a power plant via a standard power outlet) or a direct current (DC) power source (for example, various dry batteries or rechargeable batteries).

The memory module 152 may be any available volatile or non-volatile data storage device for storing any data generated during the measurement process of the displacement sensing module 130.

The communication module 153 is communicationally connected to the above-mentioned pressure control module 120, displacement sensing module 130, and scanning position control module 140 to respectively transmit control signals from the computing unit 150 to the pressure control module 120, the displacement sensing module 130, and the scanning position control module 140, or to receive information transmitted from the pressure control module 120, the displacement sensing module 130, and the scanning position control module 140 for calculation and analysis by an analysis module 158 or for storage in the memory module 152. The communication module 153 may also be communicationally connected to a cloud database, a cloud computing center, or both to obtain reference data for pulse wave interpretation and perform analysis and calculation to facilitate subsequent pulse wave interpretation. The cloud database may include, for example, an acupuncture meridian database, a pulse pattern comparison database, a Chinese herbal medicine database, a pulse wave analysis database, a vascular elasticity database, a pulse wave velocity (PWV) database, a continuous blood pressure analysis database, cloud-based artificial intelligence computing, or various other databases related to pulse wave analysis, so as to improve the accuracy of pulse wave interpretation results, but is not limited thereto. The communication module 153 may also transmit the obtained monitoring data to the cloud computing center for data analysis and calculation to obtain interpretation results.

The operation module 154 provides a user interface for the radial displacement pulse wave measuring device 100, allowing a user to issue control commands through the operation module 154 to operate the pressure control module 120, the displacement sensing module 130, and the scanning position control module 140. The user interface may also be displayed on a remote display device via the communication module 153 to facilitate remote operation required for telemedicine.

The processing module 156 may comprise a control module 157 and an analysis module 158. The control module 157 is responsible for providing control instructions to the pressure control module 120, the displacement sensing module 130, and the scanning position control module 140. The analysis module 158 is responsible for performing calculation and analysis on the information transmitted from the pressure control module 120, the displacement sensing module 130, and the scanning position control module 140. For example, the measurement signal of the radial displacement pulse wave obtained by the displacement sensing module 130 simultaneously contains a Z-axis displacement caused by pulse beats (i.e., a dynamic alternating current (AC) signal) and a Z-axis displacement caused by the downward pressing of the airbag 110 onto the measurement site 160 (i.e., a downward pressing depth along the Z-axis, which is a static direct current (DC) signal). At this point, if only the Z-axis displacement caused by the downward pressing of the airbag 110 onto the measurement site 160 is desired to be clearly measured, the analysis module 158 of the computing unit 150 may be used to filter out the dynamic Z-axis displacement caused by the pulse beats from the obtained measurement signal of the radial displacement pulse wave, leaving only the static Z-axis displacement caused by the downward pressing of the airbag 110 onto the measurement site 160. By using this static DC signal, it is convenient to determine the Z-axis downward pressing depth and the corresponding pressure value of the airbag 110 that provide a maximum signal-to-noise ratio of the pulse wave signal, which can serve as a target setting for the pressure control module 120. Therefore, the accuracy and repeatability of determining the measurement signal can be improved, facilitating the acquisition of the radial displacement pulse wave signal of the blood vessel of the subject.

The display module 155 is configured to display a user interface of the control module 157, to display information transmitted from the pressure control module 120, the displacement sensing module 130, and the scanning position control module 140 to the analysis module 158, and to display analysis results generated by the analysis module 158 based on the information. When the display module 155 is located remotely, the above-mentioned information may also be displayed on the remotely located display module 155 via the communication module 153, thereby facilitating telemedicine.

The wearing portion 200 in FIG. 2 is fixed to the housing 230. An internal space of the wearing portion 200 is configured to accommodate a limb portion 240 of the subject, where the measurement site 160 is located. Furthermore, an artery 250 passes under the skin of the measurement site 160, allowing the displacement sensing module 130 to measure a radial displacement pulse wave of the artery 250. The wearing portion 200 comprises a hard outer layer 210 and a soft inner layer 220. The hard outer layer 210 is substantially cylindrical in shape, and the soft inner layer 220 is generally formed by an extension of the airbag 110.

The hard outer layer 210 of the wearing portion 200 allows a distance MD between the displacement sensing module 130 and a farthest point 212 on the hard outer layer 210 relative to the displacement sensing module 130 to be fixed, so that a stable measurement reference can be continuously maintained during the pulse wave measurement process. Therefore, the material of the hard outer layer 210 needs to have a certain mechanical strength to ensure that the above-mentioned distance MD can still be continuously maintained as a fixed value after the wearing portion 200 is properly secured to the subject and the airbag 110 is pressurized. According to some embodiments, when the airbag 110 is distributed only on an upper half of the hard outer layer 210, it is easier to maintain the distance MD as a fixed value.

The structural design of the hard outer layer 210 may vary depending on the shape of the limb portion where the measurement site 160 of the subject is located. Therefore, FIG. 2 merely illustrates a simplified schematic diagram, and the actual structure of the hard outer layer 210 should not be limited thereto. For example, the hard outer layer 210 may adopt a design similar to a metal watch strap and clasp, allowing the length of the metal strap to be adjustable, which facilitates fitting onto the limb portion where the measurement site 160 is located and also allows for convenient storage and portability. If the hard outer layer 210 adopts a structure design of an annular sheet-like hard shell, the circumference of the hard outer layer 210 is fixed, and the limb portion where the measurement site 160 of the subject is located can directly pass through the hard outer layer 210. Alternatively, the hard outer layer 210 may adopt a structure design of two or more annular sheet-like hard shells, together with appropriate hard outer layer fasteners (such as clasps) to fix overlapping portions between different annular sheet-like hard shells. Alternatively, the hard outer layer 210 may also be designed as a C-shaped structure, allowing the wrist to be placed into the structure through an opening of the C-shape during measurement, with a soft inner layer 220 disposed on an inner side of the opening.

As described above, the soft inner layer 220 is substantially formed by the airbag 110, so the internal gas pressure of the soft inner layer 220 can also be controlled by the pressure control module 120. Accordingly, the opening size of the hard outer layer 210 can be more easily adjusted, allowing the wearing portion 200 to more conveniently accommodate limbs of different sizes where the measurement site 160 is located, thereby making the pulse wave measurement easier. Similarly, the structural design of the soft inner layer 220 may vary depending on the shape of the limb portion where the measurement site 160 of the subject is located. Therefore, FIG. 2 merely illustrates a simplified schematic diagram, and the actual structure of the soft inner layer 220 should not be limited thereto. For example, the soft inner layer 220 may be directly formed by a cylindrical airbag 110. Alternatively, the soft inner layer 220 may be formed by a sheet-like airbag 110, with two side ends of the airbag 110 overlapping and appropriately fixed by soft inner layer fasteners (such as hook-and-loop fasteners). Furthermore, the soft inner layer 220 may only retain a measurement portion 110a (including the transparent window 112 and the contact portion 114 of the airbag 110) as shown in FIG. 2, and other portions of the airbag 110 may be omitted.

According to some embodiments, in order to accommodate a wider range of sizes and shapes of the limb portion 240 where the measurement site 160 is located, in addition to the airbag 110, the soft inner layer 220 may further comprise a plurality of independent auxiliary airbags. The pressure of each auxiliary airbag can be independently controlled by the pressure control module 120, allowing each independent airbag to easily fill the remaining irregular spaces between the inner side of the wearing portion 200 and the limb portion.

### Method for Locating the Measurement Position of Radial Displacement Pulse Wave

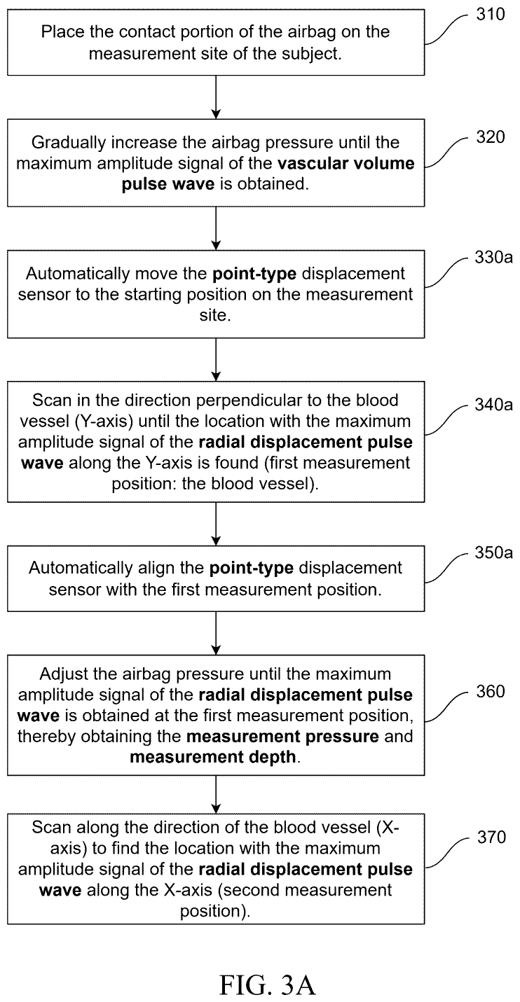

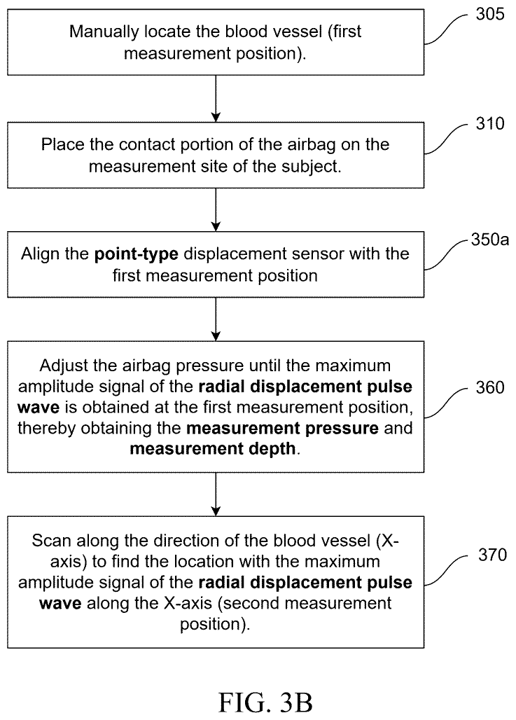

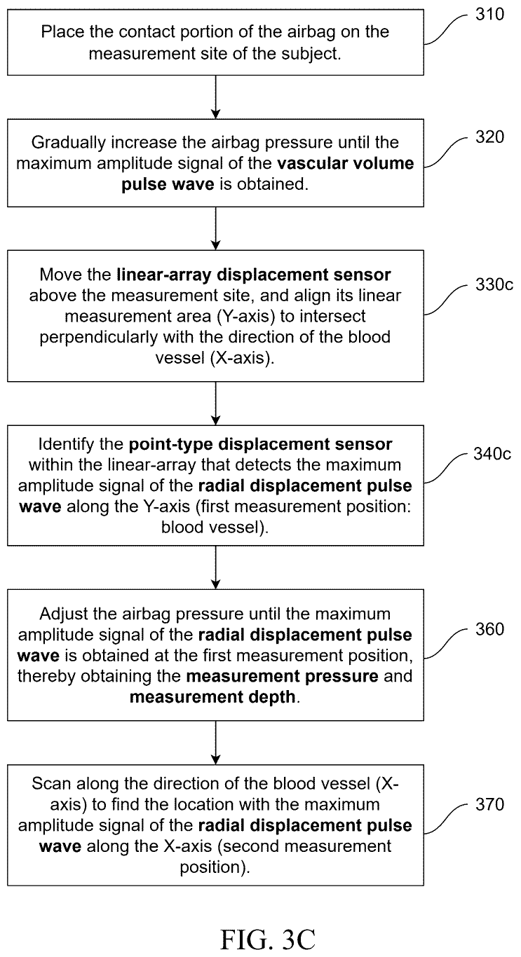

The following describes a method for measuring pulse waves using the above-mentioned radial displacement pulse wave measuring device 100. When the radial displacement pulse wave measuring device 100 includes the scanning position control module 140, under an appropriate downward pressing depth of the airbag, the movement of the displacement sensing module 130 can be automatically controlled to perform scanning over the measurement site 160 of the subject to locate an optimal measurement position within the measurement site 160. Please refer to FIGS. 3A and 3B.

FIG. 3A is a schematic diagram illustrating an operation flow for automatically locating a measurement point of a radial displacement pulse wave using the scanning position control module 140 according to one embodiment of the present disclosure, in which the displacement sensing module 130 is a point-type displacement sensor.

In step 310 of FIG. 3A, the airbag 110 of the radial displacement pulse wave measuring device 100 is first placed on the measurement site 160 of the subject, allowing the contact portion 114 of the airbag 110 to come into contact with the skin of the measurement site 160. By detecting the maximum amplitude of the vascular volume pulse wave, the downward pressing depth of the airbag 110 can be roughly adjusted to a depth at which the vascular pulse beats can be detected, even before the displacement sensing module 130 is aligned with the blood vessel, thereby accelerating the overall measurement process.

In step 320, the pressure control module 120 is used to gradually increase the internal pressure of the airbag 110 to raise the internal pressure of the airbag 110. When the pressure sensor 122 in the pressure control module 120 detects that the signal generated by the pulse beat reaches its maximum value, that is, when the amplitude of the vascular volume pulse wave reaches its maximum, a rough downward pressing depth is identified, and the pressure control module 120 stops pressurizing the airbag 110 to maintain the internal pressure of the airbag 110. According to some embodiments, step 320 may be skipped, and step 330a may be executed directly.

In step 330a, a starting position (coordinate) is first selected on the measurement site 160 of the subject. The point-type displacement sensor is then moved to a position above the starting position by the scanning position control module 140, serving as the starting point for scanning. The distance between the point-type displacement sensor and the measurement site 160 is then measured.

In step 340a, the scanning position control module 140 is used to move the point-type displacement sensor to scan along a direction perpendicular to the vascular direction, that is, along the Y-axis. Within a certain region, the amplitude of the radial displacement pulse wave gradually increases and then decreases. The position along the Y-axis where the amplitude of the radial displacement pulse wave reaches its maximum is identified, which corresponds to the location of the blood vessel, referred to as the first measurement position.

In step 350a, the scanning position control module 140 is used to align the point-type displacement sensor with the first measurement position.

In step 360, the pressure of the airbag 110 is adjusted until the maximum amplitude signal of the radial displacement pulse wave is obtained at the first measurement position, resulting in a measurement signal of the radial displacement pulse wave with an improved signal-to-noise ratio. At this point, the pressure of the airbag 110 corresponds to the optimal measurement pressure, and the downward pressing depth of the airbag 110 on the measurement site 160 corresponds to the optimal measurement depth. Therefore, upon completing step 360, the process of locating the pulse wave measurement point can be concluded.

However, if there are special requirements (for example, in traditional Chinese medicine, specific pulse diagnosis positions are emphasized) or if the detected radial displacement pulse wave has an insufficient signal-to-noise ratio, step 370 may be performed. In step 370, the scanning position control module 140 is used to scan along the vascular direction, that is, along the X-axis, to locate the position where the radial displacement pulse wave signal exhibits a local maximum (the second measurement position). Once located, the second measurement position serves as the measurement location for the radial displacement pulse wave.

When the radial displacement pulse wave measuring device 100 does not include the scanning position control module 140 or when the scanning position control module 140 is not intended to be used, the movement of the point-type displacement sensor can be manually controlled to perform scanning over the measurement site 160 of the subject, so as to locate an optimal measurement position within the measurement site 160. Since the movement of the point-type displacement sensor is manually controlled, the first half of the manual control process differs from the first half of the automatic control process. Please refer to FIG. 3B.

Next, please refer to FIG. 3B. FIG. 3B is a schematic diagram illustrating an operational flow of manually locating the measurement point of the radial displacement pulse wave according to another embodiment of the present disclosure, in which the displacement sensing module 130 is a point-type displacement sensor. Since the measurement point needs to be manually located first, it is preferable that at least the transparent window 112 and the contact portion 114 of the airbag 110 are made of transparent materials. Alternatively, the main body of the airbag 110, the transparent window 112, and the contact portion 114 may all be made of transparent materials, or openings may be formed in the visible areas of the wearable portion 200 and the housing 230, or these components may be made of transparent materials to facilitate visual identification of the measurement point by the user. In step 305, a manual operation is used to replace the function of the scanning position control module 140. After the user manually touches the artery of the measurement site 160 where the pulse is felt, the location of the artery within the measurement site 160 (the first measurement position) can be directly identified. In this step, when the contact portion 114 is transparent or semi-transparent, a reflective patch that reflects the measurement signal may be applied to the skin surface at the location of the artery. The measurement signal emitted by the displacement sensing module 130 is then aligned with the reflective patch before performing the subsequent steps 310, 350a, 360, and 370. Since the subsequent steps 310, 350a, 360, and 370 are identical or similar to the processes described in FIG. 3A, detailed descriptions are omitted here.

Next, please refer to FIG. 3C. FIG. 3C is a schematic diagram illustrating an operational flow of locating the measurement point of the radial displacement pulse wave according to another embodiment of the present disclosure, in which the displacement sensing module 130 is a linear array displacement sensor. The process shown in FIG. 3C is similar to that of FIG. 3A, except that the linear array displacement sensor is formed by arranging multiple point-type displacement sensors in a linear configuration. Therefore, the scanning process along the Y-axis for locating the first measurement position can be omitted. Accordingly, only the slightly different steps 330c and 340c are described below, and the remaining steps will not be repeated. In addition, step 320 may also be skipped, and step 330c may be performed directly to measure the radial displacement pulse wave.

In step 330c, the scanning position control module 140 or a manual operation can be used to directly align the linear measurement area of the linear array displacement sensor parallel to the Y-axis and across the location of the artery. In step 340c, the internal pressure of the airbag is gradually adjusted while observing which of the point-type displacement sensors within the linear array displacement sensor detects the maximum signal value of the radial displacement pulse wave. This allows the determination that the corresponding point-type displacement sensor is positioned above the artery, meaning that the first measurement position has been located, and the subsequent step 360 can be directly performed.

If a planar-type array displacement sensor is used, the scanning steps along the X-axis and Y-axis to locate the position with the maximum signal (i.e., the starting position and the first measurement position) can be omitted. The planar-type array displacement sensor can be directly positioned above the measurement site 160, and the point-type displacement sensor within the planar-type array displacement sensor that detects the maximum radial displacement pulse wave signal can be identified. Once the optimal signal-to-noise ratio measurement depth is found, it indicates that this point-type displacement sensor is located above the aforementioned second measurement position. Thus, the measurement signal obtained from this point-type displacement sensor can be used for subsequent applications. Therefore, when the radial displacement pulse wave measuring device 100 uses either a planar-type array displacement sensor or a linear-type array displacement sensor, the use of the scanning position control module 140 can be selected based on actual requirements.

As described above, the cooperation between the airbag 110 and the pressure control module 120 allows the airbag 110 to control the downward pressing depth along the Z-axis onto the measurement site 160, so that the displacement sensing module 130 can locate an optimal pulse wave measurement depth along the Z-axis with a better signal-to-noise ratio. Once the airbag 110 has pressed against the measurement site 160, the scanning position control module 140 enables the displacement sensing module 130 to locate an optimal pulse wave measurement position on the X-Y plane with a better signal-to-noise ratio. Therefore, the radial displacement pulse wave measuring device 100 can easily locate an optimal measurement position with a better signal-to-noise ratio within the measurement site 160 of the subject, so as to obtain an improved radial displacement pulse wave signal.

### Analysis of Pulse Waveforms in Response to Pressure Variation

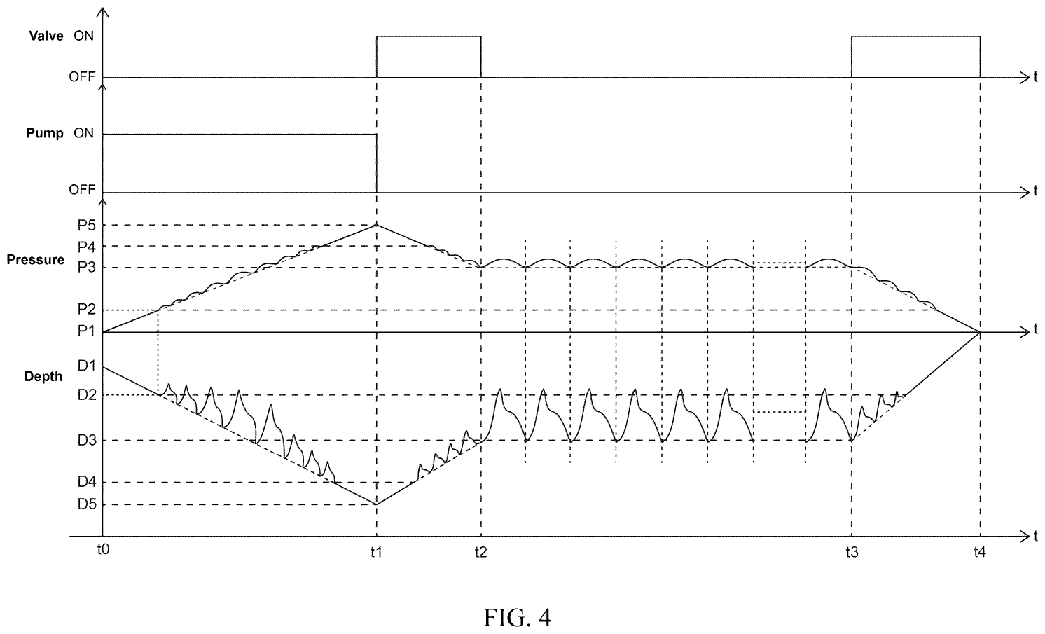

FIG. 4 is a schematic diagram illustrating waveforms of a vascular volume pulse wave and a radial displacement pulse wave varying with pressure and time, according to one embodiment of the present disclosure. In FIG. 4, the horizontal axis represents time, and the vertical axis is divided into four segments from top to bottom: valve, pump, pressure, and depth. The curve in the topmost valve segment shows the on/off status of the valve over time. The curve in the pump segment shows the on/off status of the pump over time. The curve in the pressure segment shows the real-time variation of the internal pressure of the airbag 110, which corresponds to the vascular volume pulse wave measured by the pressure sensor 122. The curve in the depth segment at the bottom shows the real-time variation of the downward pressing depth of the airbag 110 along the Z-axis onto the measurement site, which corresponds to the radial displacement pulse wave measured by the displacement sensing module 130. Please refer to both FIG. 4 and Table 1 below for a detailed description of the waveform variations during different time periods (t0-t1, t1-t2, t2-t3, and t3-t4) along the time axis.

TABLE 1
Relationship between the pressure and depth in FIG. 4 and
the corresponding radial displacement pulse wave signals.

| | | | | Amplitude Signal |
|---|---|---|---|---|
| | | | | of Radial |
| | Time | Compression | Airbag | Displacement |
| Period | Axis | Depth | Pressure | Pulse Wave |
| Period I | t0 | D1 | P1 = 0 | None |
| Period II | t1 | D5 | P5 | None |
| | | D4 | P4 | Start to appear |
| Period III | t2 | D3 | P3 | Maximum |
| Period IV | t3 | D3 | P3 | Maximum |
| | | D2 | P2 | Start to disappear |
| | t4 | 0 | P1 = 0 | None |

During Period I (t0-t1), the air valve is closed to stop deflation of the airbag 110, and the pump is turned on to inflate the airbag 110, thereby continuously and gradually increasing the internal pressure of the airbag 110. During the gradual pressurization process up to P5, it can be observed that the signals of the vascular volume pulse wave and the radial displacement pulse wave transition from absent, to appearing, then gradually increasing, subsequently decreasing, and finally disappearing. In this manner, the maximum pressure at which the airbag 110 should be deflated can be determined as P5. The operation in Period I is an optional step that may be omitted. The internal pressure of the airbag may be directly adjusted, and the variations in the amplitude of the vascular radial displacement pulse wave and the compression depth may be acquired to obtain a pulse wave signal of the vascular radial displacement pulse wave with an optimized signal-to-noise ratio.

In the second period (t1-t2), the pump is turned off at time t1 to stop inflating the airbag 110, and the valve is opened to allow the airbag 110 to gradually deflate. When the pressure of the airbag 110 decreases to P4 and the pressing depth reaches D4, signals of both the vascular volume pulse wave and the radial displacement pulse wave begin to appear. As the pressure of the airbag 110 continues to gradually decrease, at time t2, when the pressure reaches P3 and the pressing depth reaches D3, the amplitude of the radial displacement pulse wave reaches its maximum value. At this moment, the amplitude of the vascular volume pulse wave is also near its maximum value. Therefore, the pressure P3 and the pressing depth D3 are considered the optimal conditions for measuring the radial displacement pulse wave. At time t2, the valve is also turned off to maintain this state for a period of time, during which the vascular volume pulse wave, the radial displacement pulse wave, or both can be monitored and recorded as required. Since the pressure P3 and the pressing depth D3 required to achieve the maximum amplitude of the vascular volume pulse wave (which is related to the vascular volume change) and the maximum amplitude of the radial displacement pulse wave (which is related to the vascular diameter change) are not necessarily identical, the appropriate pressure P3 and pressing depth D3 to be maintained in this stage can be determined based on the actual measurement requirements.

In the third period (t2-t3), the pressure of the airbag is maintained at P3 and the pressing depth of the airbag is maintained at D3, so that the vascular volume pulse wave, the radial displacement pulse wave, or both can be monitored and recorded as required.

In the fourth period (t3 to t4), after completing the monitoring and recording performed during the third period at time t3, the valve is opened to allow the airbag 110 to gradually release pressure. When the pressure of the airbag 110 decreases to P2 and the pressing depth is D2, both the vascular volume pulse wave and the radial displacement pulse wave signals disappear. Thereafter, the airbag 110 continues to release pressure until the pressure of the airbag 110 reaches zero, marking the end time point t4.

By comparing the vascular volume pulse wave in the pressure segment and the radial displacement pulse wave in the depth segment during the third period (t2-t3), it can be observed that the radial displacement pulse wave exhibits more waveform details. Therefore, it can be used for more extensive data analysis to obtain multiple physiological parameters, enabling its application in a broader range of physiological monitoring fields. Examples will be provided below.

### Blood Pressure Measurement Method

The following describes how to perform blood pressure measurement. To measure blood pressure, the measurement site 160 of the subject may be, for example, the radial artery located on the inner side of the wrist or the brachial artery located on the inner side of the upper arm near the elbow joint.

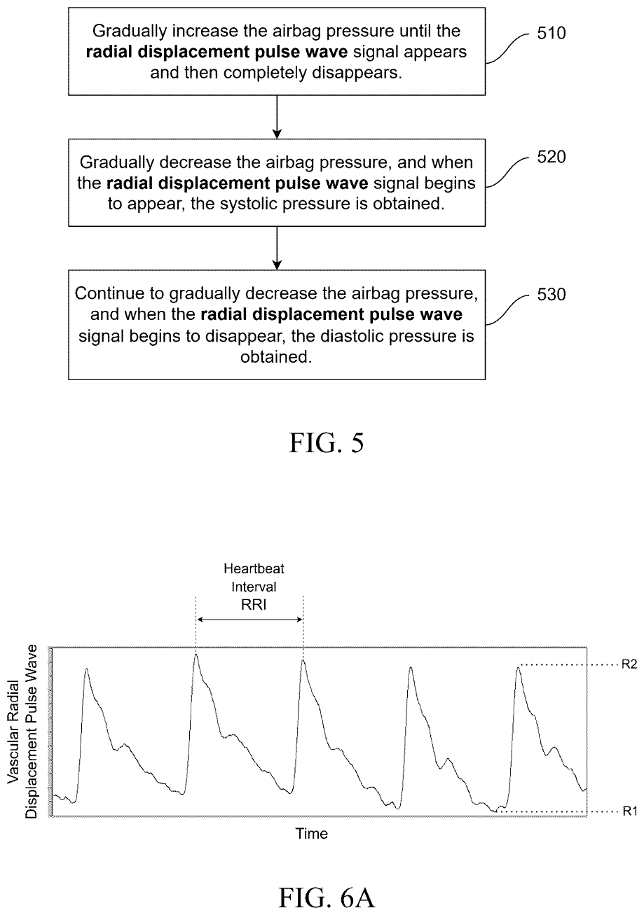

FIG. 5 is a schematic flow diagram illustrating the process of measuring blood pressure using the radial displacement pulse wave measuring device shown in FIG. 1. After the first or second measurement position of the radial displacement pulse wave is located using the aforementioned method, the displacement sensing module 130 is aligned with the first or second measurement position to perform blood pressure measurement. Although both vascular volume pulse waves and radial displacement pulse waves can be used for blood pressure measurement, the signal of the radial displacement pulse wave is clearer than that of the vascular volume pulse wave. Therefore, the following description primarily uses the radial displacement pulse wave as the main basis for determining blood pressure.

Step 510 generally corresponds to the first period (Stage I) shown in FIG. 4 and Table 1. In step 510, the pressure control module 120 gradually increases the internal pressure of the airbag 110 until the signal amplitude of the radial displacement pulse wave appears and then disappears.

Step 520 generally corresponds to the period from time t1 to time t2 during the second stage (Stage II) shown in FIG. 4 and Table 1, that is, from the moment when the airbag 110 reaches a pressure P4 and a depression depth D4, to the moment when the airbag 110 reaches a pressure P3 and a depression depth D3. In step 520, the pressure control module 120 gradually decreases the internal pressure of the airbag 110. When the signal amplitude of the radial displacement pulse wave begins to reappear, after filtering out the dynamic signal of the vascular volume pulse wave caused by the heartbeat, the static DC signal of the vascular volume pulse wave obtained by the pressure sensor 122 of the pressure control module 120 represents the internal pressure value (P4) of the airbag 110, which corresponds to the systolic blood pressure (P4) of the subject.

Step 530 generally corresponds to the period from time t3 to time t4 during the fourth stage (Stage IV) shown in FIG. 4 and Table 1, that is, from the moment when the airbag 110 reaches a pressure P3 and a depression depth D3, to the moment when the airbag 110 reaches a pressure P2 and a depression depth D2. In step 530, the pressure control module 120 continues to gradually decrease the internal pressure of the airbag 110. When the signal amplitude of the radial displacement pulse wave begins to disappear again, after filtering out the dynamic signal of the vascular volume pulse wave caused by the heartbeat, the static DC signal of the vascular volume pulse wave obtained by the pressure sensor 122 of the pressure control module 120 represents the internal pressure value (P2) of the airbag 110, which corresponds to the diastolic blood pressure (P2) of the subject.

### Heart Rate Variability Measurement and Application

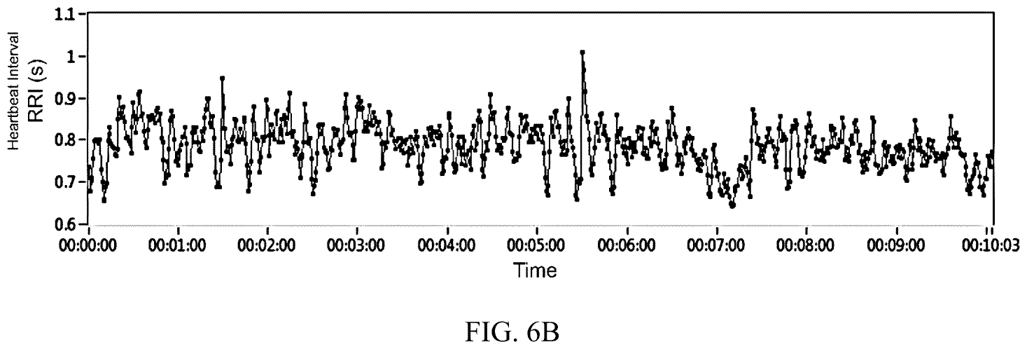

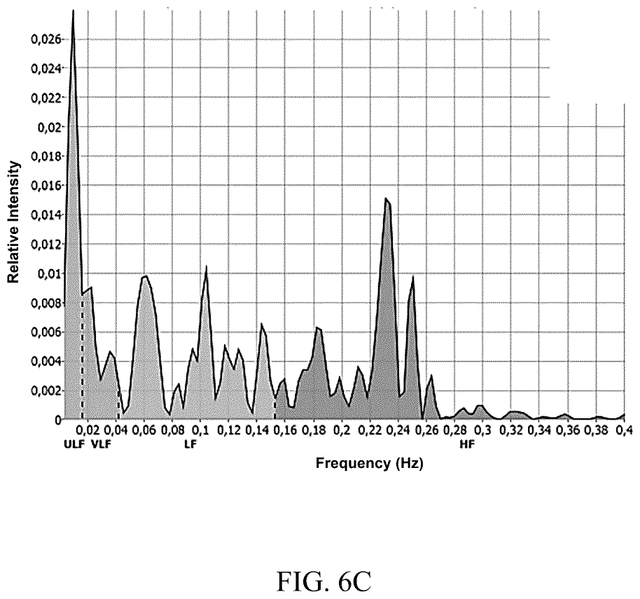

FIG. 6A is an enlarged schematic diagram of the radial displacement pulse wave during the third stage (Stage III) from time t2 to t3 shown in FIG. 4. In FIG. 6A, since the peak positions of the radial displacement pulse wave are clearly identifiable, these peak positions can be used to simulate the R-wave peaks in a conventional electrocardiogram (ECG) for extracting beat-to-beat interval (i.e. RR interval, RRI) data. By recording the radial displacement pulse wave signals for one minute and calculating the number of detected peaks, the heart rate per minute can be obtained. In FIG. 6A, the interval between two adjacent peaks on the radial displacement pulse wave curve represents the beat-to-beat interval.

Heart rate variability (HRV) refers to the variability in the time intervals between consecutive heartbeats. Under normal conditions, heartbeats do not occur at perfectly uniform intervals but exhibit slight variations, which are known as heart rate variability. Therefore, HRV analysis is a method for measuring the degree of variation in continuous beat-to-beat intervals. When using the radial displacement pulse wave measuring device 100 to measure heart rate variability, the measurement area 160 may be selected from a region with relatively thin subcutaneous tissue, such as the styloid process of the wrist and the anterior or posterior side thereof, the earlobe or the auricular artery, the palmar artery, the sole of the foot, or the toes, where a blood vessel can be located to obtain a radial displacement pulse wave with a more distinct peak.

The method for heart rate variability (HRV) analysis can be divided into two types. The first type is the time domain analysis method, which typically involves continuously measuring electrocardiogram (ECG) waveforms and directly calculating and analyzing the dispersion of the corresponding beat-to-beat intervals. Common examples include:
SDNN (Standard Deviation of NN Intervals): typically refers to the standard deviation of normal beat-to-beat intervals over a 24-hour period, expressed in milliseconds (ms).
SDANN (Standard Deviation of the Average NN Intervals): typically refers to dividing the continuous recording into five-minute segments, calculating the average NN interval for each segment, and then computing the standard deviation of these average intervals; expressed in milliseconds (ms).

The following provides a more detailed explanation using SDNN as an example. SDNN is a measure of the overall activity of the autonomic nervous system, reflecting the combined activity of the sympathetic and parasympathetic nervous systems, and represents the body's ability to regulate physiological processes. SDNN is the standard deviation of the RR intervals (RRI) within a specific time period, since each RRI is not necessarily identical. The unit of SDNN is milliseconds (ms). The standard deviation of the RRI describes the degree of dispersion of each RRI around their average value. The greater the dispersion, the larger the standard deviation. Conversely, if all RRIs are identical, the standard deviation of the RRI will be 0. After calculating all RRIs from the HRV recording, the relationship between the RR intervals (RRI) and time can be obtained, as shown in FIG. 6B. The SDNN can be calculated using Equations (1) and (2) below.

$$SDNN = \sqrt{\frac{1}{N - 1} {\sum }_{i = 1}^{N} {( {RR}_{i} - \overline{RR} )}^{2}} \qquad ( 1 )$$

$$\overline{RR} = \frac{1}{N} {\sum }_{i = 1}^{N} {RR}_{i + 1} \qquad ( 2 )$$

Where RRi represents the i-th RR interval (RRI), N represents the total number of measured RRIs, and R̅R̅ represents the average value of all RRIs. The variable i is a positive integer.

The second method is the frequency domain analysis, which utilizes discrete Fourier transform (DFT) to convert the time series of beat-to-beat intervals into the frequency domain, and expresses the result in terms of power spectral density or spectral distribution, as shown in FIG. 6C. Typically, spectral analysis of heart rate variability (HRV) signals requires a stable recording of 200 to 500 consecutive RRIs, which corresponds to several minutes of recording time. The RRI spectrum generally falls below 1 Hz, for example, several RRI spectral peaks can be observed within the range of 0 to 0.4 Hz, including the ultra-low frequency (ULF: ≤0.003 Hz), very-low frequency (VLF: 0.0033-0.04 Hz), low frequency (LF: 0.04-0.15 Hz), and high frequency (HF: 0.15-0.40 Hz) bands. Among them, the HF band usually reflects parasympathetic nervous system activity, the LF band is influenced by both the sympathetic and parasympathetic nervous systems, and the LF/HF ratio reflects the balance between sympathetic and parasympathetic nervous system activities.

### Pulse Wave Velocity Measurement Method

Pulse Wave Velocity (PWV) refers to the speed at which the fluctuations of arterial blood propagate through the blood vessels. These fluctuations are caused by the pressure waves generated during ventricular contraction of the heart, which propagate along the arterial system. Therefore, PWV can reflect the elasticity and degree of stiffness of the arteries. PWV is inversely proportional to the viscoelasticity of the arterial walls, meaning that the stiffer the arteries, the faster the PWV; conversely, the softer the arteries, the slower the PWV.

The interpretation of PWV should take into account factors such as age, gender, diseases, and risk factors. Generally, the normal PWV of the aorta is approximately 5-7 m/s, while a PWV greater than 10 m/s indicates significant arterial stiffness. PWV is closely related to the incidence and mortality of cardiovascular diseases; therefore, PWV can serve as an important indicator for assessing cardiovascular risk and prognosis, including conditions such as coronary artery disease, arteriosclerosis, or connective tissue diseases.

The measurement of PWV is primarily performed by simultaneously recording the pressure waveforms at two different locations along the arterial tree. These two locations include a proximal site near the heart and a distal site farther from the heart. By simultaneously recording the waveforms at both the proximal and distal sites, the time delay ΔT (=T₂−T₁) of the distal waveform relative to the proximal waveform can be directly measured. After measuring the distance ΔD between the proximal and distal sites, the PWV can be calculated according to Equation (3) below.

$$PWV = Δ D / Δ T \qquad ( 3 )$$

There are two methods for measuring the delay time ΔT. The first method uses two displacement sensing modules 130, and the second method uses an electrocardiogram (ECG) in combination with a single displacement sensing module 130.

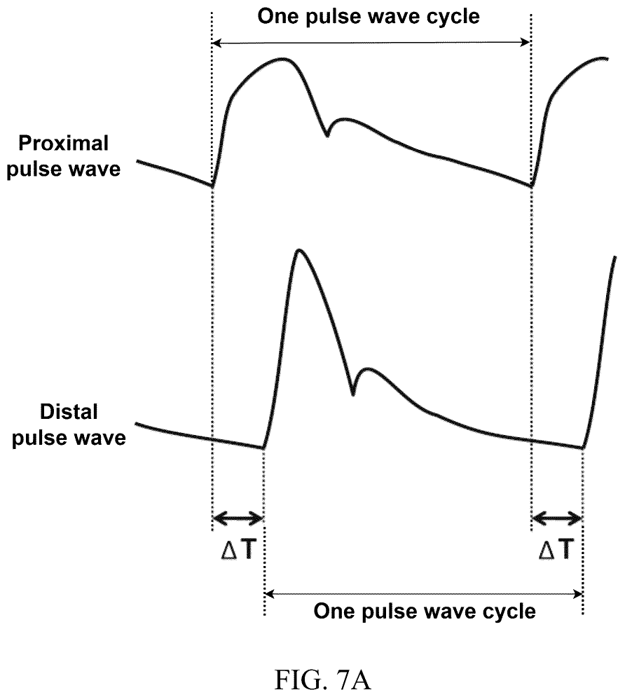

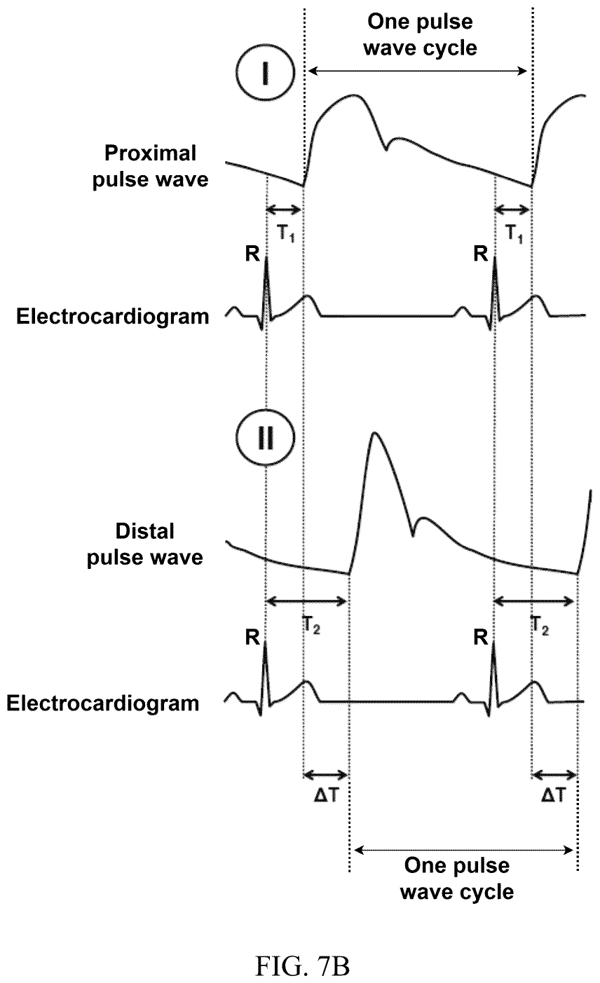

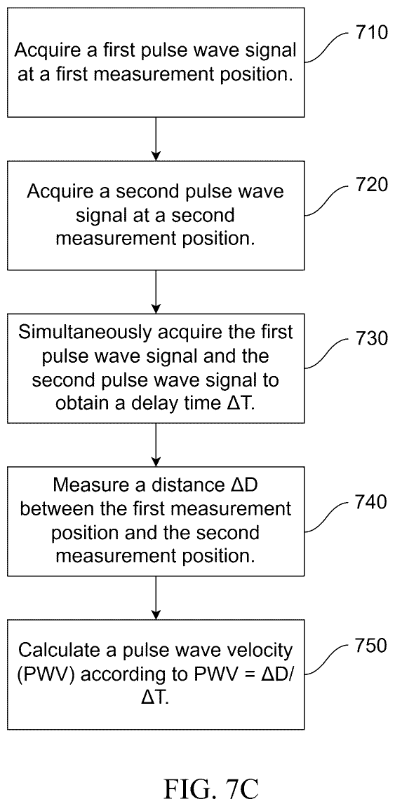

FIG. 7A is a schematic diagram of simultaneously using two pulse wave measuring devices to measure pulse wave velocity according to one embodiment of the present disclosure. In FIG. 7A, two radial displacement pulse wave measuring devices 100 are directly used to simultaneously record the proximal radial displacement pulse wave and the distal radial displacement pulse wave, allowing direct measurement of the delay time ΔT between the proximal and distal radial displacement pulse waves, based on either valley-to-valley or peak-to-peak alignment. FIG. 7A shows the valley-to-valley delay time ΔT. If either of the two radial displacement pulse wave measuring devices 100 uses a manually aligned displacement sensing module 130 to align with the measurement site 160, the scanning position control module 140 can be omitted, leaving only the airbag 110, the pressure control module 120, the displacement sensing module 130, and the computing unit 150 in the radial displacement pulse wave measuring device 100. In addition, the two radial displacement pulse wave measuring devices 100 can share a single computing unit 150, thereby allowing one of the computing units 150 to be omitted.

FIG. 7B is a schematic diagram of measuring pulse wave velocity with the assistance of an electrocardiogram (ECG) according to an embodiment of the present disclosure. In FIG. 7B, the R-wave peak of the ECG is used as a timing reference to align the proximal and distal pulse waves. In the upper part (I) of FIG. 7B, the delay time of the valley of the proximal radial displacement pulse wave relative to the R-wave peak of the ECG is defined as T₁. In the lower part (II) of FIG. 7B, the delay time of the valley of the distal radial displacement pulse wave relative to the R-wave peak of the ECG is defined as T₂. The time difference between T₁ and T₂ is defined as the delay time ΔT. The delay time ΔT measured by the above two methods represents the pulse transit time (PTT).

The distance ΔD between the proximal and distal sites can be directly measured using a flexible tape measure to determine the distance between the two measurement positions. Alternatively, the sum of the distances from the heart to each of the two measurement positions can be measured and multiplied by a coefficient. The proximal and distal sites may include, for example, the carotid artery, femoral artery, brachial artery, radial artery, ankle artery, digital artery, or posterior tibial artery, and the measurement positions are set according to the specific measurement requirements. Different combinations of proximal and distal positions allow the calculation of different pulse wave velocities.

FIG. 7C is a flowchart illustrating the pulse wave velocity measurement method using the approach shown in FIG. 7A.

In step 710, one of the radial displacement pulse wave measuring devices 100 is placed at the first measurement position of the proximal artery. The device is aligned with the target artery to be measured, either automatically or manually, following the operation process described in FIG. 3A or FIG. 3B. The vascular radial displacement pulse wave at the first measurement position of the artery is then captured.

In step 720, another radial displacement pulse wave measuring device 100 is placed at the second measurement position of the distal artery. The device is aligned with the target artery to be measured, either automatically or manually, following the operation process described in FIG. 3A or FIG. 3B. The vascular radial displacement pulse wave at the second measurement position of the artery is then captured.

In step 730, the vascular radial displacement pulse wave signals obtained in steps 710 and 720 are simultaneously acquired to obtain the pulse wave delay time ΔT. This step 730 may be repeated multiple times within a certain period to obtain the successive delay times ΔTi for each pulse wave during that period, that is, the successive pulse transit times (PTTi).

In step 740, the distance ΔD between the first measurement position and the second measurement position is measured. For example, different displacement sensors such as point-type, linear matrix-type, or planar matrix-type sensors may be used to measure the distance ΔD. When using point-type displacement sensors, the distance ΔD can be obtained by using a measuring tape to measure the length between the two point-type displacement sensors placed at the first and second measurement positions, respectively.

According to some embodiments, when a linear or planar optical displacement sensor is used, only one optical displacement sensor is required to simultaneously measure the radial displacement pulse waves at two positions by utilizing two non-adjacent sensing units within the optical displacement sensor with a known distance between them. Since the measurement signal of the optical displacement sensor is typically a laser beam, by configuring the scanning range of the optical displacement sensor to cover both the first and second measurement positions, the distance between these two positions within the scanning range can be estimated by using the two non-adjacent sensing units with a known distance and applying simple geometric proportional calculations.

In step 750, based on the successive pulse transit times (PTTi) obtained in step 730 and the distance ΔD obtained in step 740, the successive pulse wave velocities are calculated using the formula: ΔD/ΔTi=ΔD/PTTi.

### Continuous Blood Pressure Measurement

Currently, commonly used cuff-based blood pressure monitors on the market determine systolic and diastolic pressure by detecting vascular volume pulse waves. Although convenient, these devices provide inaccurate measurements and cannot offer real-time blood pressure monitoring. In addition, wearable continuous blood pressure monitors typically estimate blood pressure based on the correlation between pulse transit time (PTT) and blood pressure, using the Bramwell-Hill formula. However, conventional methods of calculating continuous blood pressure using the Bramwell-Hill formula suffer from inaccuracies in the real-time estimation of vascular diameter and insufficient sensitivity in pulse wave measurement. These shortcomings lead to errors in the calculation of pulse transit time, resulting in inaccurate continuous blood pressure estimation derived from the Bramwell-Hill formula. Accordingly, an embodiment of the present disclosure provides a continuous blood pressure measurement device capable of improving the accuracy of vascular diameter measurement and enhancing the sensitivity of pulse wave detection, thereby increasing the measurement accuracy of wearable continuous blood pressure monitors.

The Bramwell-Hill formula is a mathematical model that describes the relationship between vascular diameter variation and pulse wave velocity (PWV). This formula is a modification of the Moens-Korteweg equation, taking into account the reduction in arterial compliance with increasing pressure and the increase in vascular volume (arterial dilation) as pressure rises. The Bramwell-Hill formula is expressed as follows in Equation (4):

$$Δ P = {ρ ( \frac{Δ D}{PTT} )}^{2} ( \frac{Δ R}{R} ) = ρ \times {PWV}^{2} ( \frac{Δ R}{R} ) \qquad ( 4 )$$

Where ΔP represents the blood pressure variation, p is the blood density, ΔD is the distance between the first measurement position and the second measurement position in the above-mentioned pulse wave velocity measurement, PTT is the pulse transit time from the first measurement position to the second measurement position, ΔR is the change in the arterial diameter at the first or second measurement position on the wrist, and R is the arterial diameter at the first or second measurement position on the wrist as a reference. Here, (ΔD/PTT) represents the pulse wave velocity (PWV).

Therefore, by using a displacement sensor to measure the arterial diameter variation ΔRi for each pulse wave and employing the pulse wave velocity (PWV) measurement method to obtain the PWVi of each pulse wave, the Bramwell-Hill formula can be applied to estimate the blood pressure variation ΔPi for each pulse wave, thereby achieving the purpose of continuous blood pressure measurement. The continuous blood pressure measurement method is described in detail below.

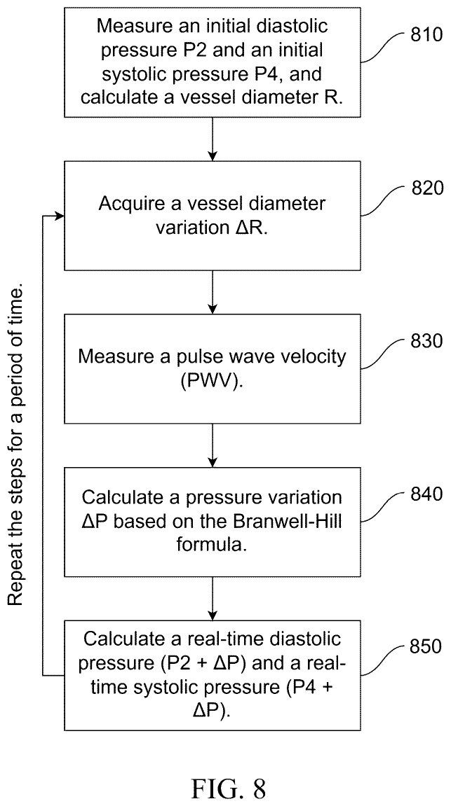

FIG. 8 is a flowchart of a method for continuously measuring blood pressure.

In step 810, the initial diastolic pressure and initial systolic pressure are first measured. According to the blood pressure measurement method described in the previous section, the subject's initial diastolic pressure P2 and initial systolic pressure P4 are obtained. Additionally, the first pressing depth D2 of the airbag 110 on the measurement site 160 when the initial diastolic pressure P2 occurs, and the second pressing depth D4 of the airbag 110 on the measurement site 160 when the initial systolic pressure P4 occurs, are measured, as shown in FIG. 4 and Table 1. The vascular diameter R can then be obtained by calculating the difference between the second pressing depth and the first pressing depth, i.e., R=(D4−D2).

In step 820, the vascular diameter and the vascular diameter variation are calculated. As shown in FIG. 6A, the waveform of the vascular radial displacement pulse wave varies over time. The difference between the peak position (R2) and the valley position (R1) of each radial displacement pulse wave represents the vascular diameter variation ΔR caused by each heartbeat.

In step 830, the pulse wave velocity (PWV) of each pulse wave is measured according to the pulse wave measurement method described in the aforementioned section.

In step 840, based on the obtained vascular diameter variation ΔR, vascular diameter R, and pulse wave velocity (PWV), along with the subject's blood density, the Bramwell-Hill equation is used to calculate the blood pressure variation ΔP.

In step 850, based on the initial diastolic pressure P2 and initial systolic pressure P4 measured in step 810, along with the blood pressure variation ΔP obtained in step 840, the real-time diastolic pressure (P2+ΔP) and real-time systolic pressure (P4+ΔP) can be determined.

Subsequently, steps 820 to 850 are repeatedly performed over a period of time. In this way, highly accurate continuous blood pressure measurement can be achieved, thereby enabling continuous blood pressure monitoring.

As described above, by means of the cooperative design of the airbag, the pressure control module, and the displacement sensing module, the radial displacement pulse wave measuring device provided in the present disclosure is capable of directly measuring the waveform of the vascular radial displacement pulse wave. Compared with conventional methods for measuring pressure pulse waves, this direct measurement approach provides more accurate results.

Moreover, the radial displacement pulse wave measuring device has a measurement accuracy of less than 100 micrometers, enabling the acquisition of detailed pulse wave characteristics, such as waveform shape, amplitude, and temporal features. High-accuracy measurement results are extremely valuable for various pulse wave analysis applications. In addition to the commonly measured parameters such as blood pressure, heart rate variability, and pulse wave velocity, the radial displacement pulse wave measuring device is also capable of performing many other analytical applications. For example, the device can be used to assess the degree of arterial stiffness, detect vascular elasticity, monitor cardiovascular disease risk, and study hemodynamics. Such multifunctional application potential allows the radial displacement pulse wave measuring device to be widely applied in the medical and biomedical fields.

In addition, using the above-described radial displacement pulse wave measuring device to measure pulse waves is a non-invasive measurement method. Therefore, the device can be conveniently used to continuously monitor the pulse wave variations of a subject in real time, providing immediate measurement results and waveform diagrams. Furthermore, with the wearable structure design, the subject's pulse waves can be monitored continuously and stably over an extended period of time. This is highly valuable for physicians and researchers, as it helps them gain a better understanding of the subject's cardiovascular condition and make corresponding diagnostic and treatment decisions, thereby supporting improved personalized healthcare.

In summary, the above-described radial displacement pulse wave measuring device offers several advantages, including direct measurement of vascular radial displacement pulse waves, high measurement accuracy, multiple application analyses, non-invasive operation, and real-time monitoring. These advantages enable the device to be widely applied in the fields of cardiovascular medicine and biomedical research.

What is claimed is:

1. A radial displacement pulse wave measuring device for measuring a vascular radial displacement pulse wave of an artery at a measurement site of a subject, the device comprising:
a housing having a transparent portion;
an airbag disposed beneath the transparent portion of the housing and comprising a transparent window and a contact portion, wherein the transparent window overlaps the transparent portion and the contact portion is configured to be in close contact with skin of the measurement site;
a pressure control module pneumatically connected to the airbag via a gas pipeline, the pressure control module comprising a pressure sensor configured to sense an internal pressure of the airbag and a pressure adjustment module configured to adjust the internal pressure of the airbag, wherein the pressure sensor is configured to measure a vascular volumetric pulse wave of the artery;
a displacement sensing module disposed above the transparent portion and configured to emit and receive a measurement signal that penetrates the transparent portion of the housing and the transparent window of the airbag, thereby measuring a dynamic distance between the displacement sensing module and the measurement site;
a computing unit communicatively connected to the pressure control module and the displacement sensing module, the computing unit being configured to:
(i) control the pressure adjustment module to incrementally increase the internal pressure of the airbag to identify a first pressure value when a vascular volumetric pulse wave signal output by the pressure sensor is maximal;
(ii) control the pressure adjustment module to maintain the internal pressure of the airbag at the first pressure value and, while the displacement sensing module and the measurement site move relative to each other in a direction substantially perpendicular to an orientation of the artery, perform a first distance measuring scan to identify a first measurement position where a vascular radial displacement pulse wave signal output by the displacement sensing module is maximal, the first measurement position corresponding to a location of the artery;
(iii) control the pressure adjustment module to adjust the internal pressure of the airbag based on the first measurement position and to identify a second pressure value when the vascular radial displacement pulse wave signal output by the displacement sensing module is maximal; and
(iv) control the pressure adjustment module to maintain the internal pressure of the airbag at the second pressure value to maintain a pressing depth of the airbag on the first measurement position, thereby enabling the displacement sensing module to perform measurement of the vascular radial displacement pulse wave.

2. The radial displacement pulse wave measuring device of claim 1, wherein at least one of the transparent portion of the housing and the transparent window of the airbag is a transparent plate.

3. The radial displacement pulse wave measuring device of claim 2, wherein the transparent window of the airbag is the transparent plate when the transparent portion of the housing is a hole.

4. The radial displacement pulse wave measuring device of claim 1, further comprising an anti-reflection coating disposed on an outer surface of the transparent window of the airbag.

5. The radial displacement pulse wave measuring device of claim 1, further comprising a reflective layer or a dichroic layer disposed on an inner surface of the contact portion.

6. The radial displacement pulse wave measuring device of claim 1, wherein the displacement sensing module further comprises a filter configured to filter noise from the measurement signal to improve a signal-to-noise ratio of the vascular radial displacement pulse wave signal.

7. The radial displacement pulse wave measuring device of claim 1, wherein the pressure adjustment module comprises a pump and a pulse width modulation circuit for adjusting a rotation speed of the pump to achieve rapid and precise regulation of the internal pressure of the airbag.

8. The radial displacement pulse wave measuring device of claim 1, wherein the displacement sensing module comprises a photoelectric displacement sensor.

9. The radial displacement pulse wave measuring device of claim 8, wherein the photoelectric displacement sensor comprises a distance measuring device selected from a group consisting of a laser displacement meter, a fiber-optic displacement sensor, a three-dimensional laser scanner, a time-of-flight (TOF) distance sensor, a three-dimensional time-of-flight (3D TOF) distance array sensor, a laser Doppler anemometer, a laser Doppler velocimeter, a laser Doppler vibrometer, a Michelson interferometer, and a laser interferometer.

10. The radial displacement pulse wave measuring device of claim 1, further comprising a wearing portion configured to stably accommodate a limb portion of the subject at the measurement site within an internal space of the wearing portion, the wearing portion comprising:
a soft inner layer comprising the airbag; and
a hard outer layer disposed outside the soft inner layer and cooperating with the soft inner layer to form the internal space, the hard outer layer being configured during measurement of the vascular radial displacement pulse wave such that the distance between the displacement sensing module and a position on the hard outer layer farthest from the displacement sensing module remains fixed.

11. The radial displacement pulse wave measuring device of claim 10, wherein the soft inner layer further comprises a plurality of auxiliary airbags whose internal pressures are controlled by the pressure control module.

12. The radial displacement pulse wave measuring device of claim 1, further comprising a scanning position control module communicatively connected to the computing unit and configured to control the displacement sensing module to automatically perform the first distance measuring scan on the measurement site.

13. The radial displacement pulse wave measuring device of claim 12, wherein the scanning position control module comprises a single axis position controller or a dual axis position controller.

14. A method for optimizing measurement conditions for vascular radial displacement pulse wave measurement, using the radial displacement pulse-wave measuring device of claim 1, the method comprising:
(a) placing the airbag on the measurement site;
(b) incrementally adjusting the internal pressure of the airbag via the pressure control module to identify the first pressure value when the vascular volumetric pulse wave signal output by the pressure sensor is maximal;
(c) controlling, via the computing unit, the pressure adjustment module to maintain the internal pressure of the airbag at the first pressure value and, while the displacement sensing module and the measurement site move relative to each other in a direction substantially perpendicular to an orientation of the artery, performing a first distance measuring scan to identify the first measurement position where the vascular radial displacement pulse-wave signal output by the displacement sensing module is maximal, the first measurement position corresponding to a location of the artery;
(d) controlling, via the computing unit, the pressure adjustment module to adjust the internal pressure of the airbag based on the first measurement position and to identify a second pressure value when the vascular radial displacement pulse wave signal output by the displacement sensing module is maximal; and
(e) controlling, via the computing unit, the pressure adjustment module to maintain the internal pressure of the airbag at the second pressure value and maintain the pressing depth of the airbag at the first measurement position to perform measurement of the vascular radial displacement pulse wave, wherein the first measurement position, the second pressure value and the pressing depth constitute a first set of measurement conditions for measuring the vascular radial displacement pulse wave of the artery.

15. The method of claim 14, wherein the radial displacement pulse-wave measuring device further comprises a scan position control module communicatively connected to the computing unit and configured to control the displacement sensing module to automatically perform the first distance measuring scan on the measurement site.

16. The method of claim 14, wherein the displacement sensing module comprises a point-type displacement sensor.

17. The method of claim 14, wherein the displacement sensing module comprises a matrix-type displacement sensor including a plurality of point-type displacement sensors, the matrix-type displacement sensor covering a region above the artery at the measurement site, and the computing unit identifies the first measurement position as the location of the point-type displacement sensor in the matrix-type displacement sensor that outputs the maximal vascular radial displacement pulse wave signal during the first distance measuring scan.

18. The method of claim 14, further comprising:
(f) collecting vascular radial displacement pulse wave signals using the first set of measurement conditions; and
(g) analyzing the collected signals to calculate heart rate variability (HRV).

19. The method of claim 18, wherein heart rate variability (HRV) is obtained by calculating the standard deviation of 24-hour normal-to-normal RR intervals (RRI) (SDNN) according to the following equations:
$$SDNN = \sqrt{\frac{1}{N - 1} {\sum }_{i = 1}^{N} {( {RR}_{i} - \overline{RR} )}^{2}} \qquad ( 1 )$$
$$\overline{RR} = \frac{1}{N} {\sum }_{i = 1}^{N} {RR}_{i + 1} \qquad ( 2 )$$
wherein RRᵢ is the i-th RR interval,
N is the total number of RR intervals measured, and
R̅R̅ is the average of all RR intervals.

20. The method of claim 14, further comprising:
(h) moving the displacement sensing module and the measurement site relative to each other along the orientation of the artery to perform a second distance measuring scan;
(i) identifying a second measurement position at which a local maximum vascular radial displacement pulse wave signal of the artery is detected; and
(j) using the second measurement position in place of the first measurement position to establish a second set of measurement conditions for measuring the vascular radial displacement pulse wave of the artery.

21. A blood pressure measurement method, comprising:
(a) using the measurement condition optimization method of claim 14 to identify the first measurement position;
(b) measuring a vascular radial displacement pulse wave at the first measurement position;
(c) controlling, via the computing unit, the pressure adjustment module to gradually increase the internal pressure of the airbag until the vascular radial displacement pulse wave of the artery disappears;
(d) gradually releasing the internal pressure of the airbag until the vascular radial displacement pulse wave reappears, the internal pressure of the airbag upon reappearance representing the systolic blood pressure of the artery; and
(e) further gradually releasing the internal pressure of the airbag until the vascular radial displacement pulse wave disappears again, the internal pressure of the airbag upon disappearance representing the diastolic blood pressure of the artery.

22. A method for measuring pulse wave velocity, comprising
(a) continuously recording an electrocardiogram of the subject;
(b) using the measurement condition optimization method of claim 14 to identify and apply the first measurement conditions to measure and record a proximal radial displacement pulse wave at a proximal arterial site closer to the heart of the subject, thereby obtaining a delay time T₁ of the proximal radial displacement pulse wave relative to an R-wave of the electrocardiogram;
(c) using the measurement condition optimization method of claim 14 to identify and apply the first measurement conditions to measure and record a distal radial displacement pulse wave at a distal arterial site farther from the heart of the subject, thereby obtaining a delay time T₂ of the distal radial displacement pulse wave relative to an R-wave of the electrocardiogram;
(d) calculating a propagation time difference ΔT=T₂−T₁;
(e) measuring a distance ΔD between the proximal arterial site and the distal arterial site; and
(f) calculating a pulse wave velocity PWV =AD/AT.

23. A method for measuring pulse wave velocity, comprising:
(a) using the measurement condition optimization method of claim 14, wherein the radial displacement pulse-wave measuring device further comprises a second displacement sensing module;
(b) controlling, via the computing unit, the displacement sensing module and the second displacement sensing module to simultaneously measure and record a proximal radial displacement pulse wave at a proximal measurement position closer to the heart and a distal radial displacement pulse wave at a distal measurement position farther from the heart;
(c) calculating a delay time difference AT between the proximal radial displacement pulse wave and the distal radial displacement pulse wave;
(d) measuring a distance ΔD between the proximal arterial site and the distal arterial site; and
(e) calculating a pulse wave velocity PWV=ΔD/ΔT.

24. A method for continuously measuring blood pressure, comprising:
(a) measuring, via the blood pressure measurement method of claim 21, a diastolic blood pressure in an initial state and a first pressing depth of the airbag at the first measurement position corresponding thereto, and a systolic blood pressure in the initial state and a second pressing depth of the airbag at the first measurement position corresponding thereto, wherein a difference between the first pressing depth and the second pressing depth equals a vascular diameter R of the subject;
(b) calculating a vascular diameter change ΔR from a waveform of the subject's vascular radial displacement pulse wave over time;
(c) measuring the pulse wave velocity PWV of the subject using the method for measuring pulse wave velocity;
(d) obtaining a blood density ρ of the subject;
(e) calculating a blood pressure change ΔP using the Bramwell-Hill equation:
$$Δ P = ρ \times {PWV}^{2} ( \frac{Δ R}{R} ) ;$$
and
(f) calculating a real-time systolic blood pressure as the initial systolic blood pressure plus ΔP and a real-time diastolic blood pressure as the initial diastolic blood pressure plus ΔP.

25. A radial displacement pulse wave measuring device for measuring a vascular radial displacement pulse wave of an artery at a measurement site of a subject, the device comprising:
a housing having a transparent portion;
an airbag disposed beneath the transparent portion of the housing and comprising a transparent window and a contact portion, wherein the transparent window overlaps the transparent portion and the contact portion is configured to be in close contact with skin of the measurement site;
a pressure control module pneumatically connected to the airbag via a gas pipeline, the pressure control module comprising a pressure sensor configured to sense an internal pressure of the airbag and a pressure adjustment module configured to adjust the internal pressure of the airbag, wherein the pressure sensor is configured to measure a vascular volumetric pulse wave of the artery;
a displacement sensing module disposed above the transparent portion and configured to emit and receive a measurement signal that penetrates the transparent portion of the housing and the transparent window of the airbag, thereby measuring dynamic changes in distance between the displacement sensing module and the measurement site to obtain a vascular radial displacement pulse wave;
a computing unit communicatively connected to the pressure control module and the displacement sensing module, configured to respectively transmit control signals to the pressure control module and the displacement sensing module, and respectively receive information transmitted from the pressure control module and the displacement sensing module for performing calculations; and
a wearing portion disposed below the housing, and configured to stably accommodate a limb portion of the subject at the measurement site within an internal space of the wearing portion, the wearing portion comprising:
a soft inner layer comprising the airbag; and
a hard outer layer disposed outside the soft inner layer and cooperating with the soft inner layer to form the internal space, the hard outer layer being configured during measurement of the vascular radial displacement pulse wave such that the distance between the displacement sensing module and a position on the hard outer layer farthest from the displacement sensing module remains fixed.

26. The radial displacement pulse wave measuring device of claim 25, further comprising an anti-reflection coating disposed on an outer surface of the transparent window of the airbag.

27. The radial displacement pulse wave measuring device of claim 25, further comprising a reflective layer or a dichroic layer disposed on an inner surface of the contact portion.

28. The radial displacement pulse wave measuring device of claim 25, wherein the displacement sensing module further comprises a filter configured to filter noise from the measurement signal to improve a signal-to-noise ratio of the vascular radial displacement pulse wave signal.

29. The radial displacement pulse wave measuring device of claim 25, wherein the pressure adjustment module comprises a pump and a pulse width modulation circuit for adjusting a rotation speed of the pump.

30. The radial displacement pulse wave measuring device of claim 25, wherein the displacement sensing module comprises a photoelectric displacement sensor.

31. The radial displacement pulse wave measuring device of claim 30, wherein the photoelectric displacement sensor comprises a distance measuring device selected from a group consisting of a laser displacement meter, a fiber-optic displacement sensor, a three-dimensional laser scanner, a time-of-flight (TOF) distance sensor, a three-dimensional time-of-flight (3D TOF) distance array sensor, a laser Doppler anemometer, a laser Doppler velocimeter, a laser Doppler vibrometer, a Michelson interferometer, and a laser interferometer.

32. The radial displacement pulse wave measuring device of claim 25, further comprising a scanning position control module communicatively connected to the computing unit and configured to control the displacement sensing module to automatically perform a distance measuring scan on the measurement site.

33. The radial displacement pulse wave measuring device of claim 32, wherein the scanning position control module comprises a single axis position controller or a dual axis position controller.

34. A radial displacement pulse wave measuring device for measuring a vascular radial displacement pulse wave of an artery at a measurement site of a subject, the device comprising:
an airbag comprising a transparent window and a contact portion, wherein the contact portion is configured to be in close contact with skin of the measurement site;
a pressure control module pneumatically connected to the airbag via a gas pipeline, the pressure control module comprising a pressure sensor configured to sense an internal pressure of the airbag and a pressure adjustment module configured to adjust the internal pressure of the airbag;
a displacement sensing module configured to emit and receive a measurement signal that penetrates the transparent window of the airbag to measure a dynamic distance between the displacement sensing module and the measurement site;
a computing unit communicatively connected to the pressure control module and the displacement sensing module, the computing unit being configured to:
(i) control the pressure adjustment module to incrementally increase the internal pressure of the airbag to identify a first pressure value when a vascular radial displacement pulse wave signal output by the displacement sensing module is maximal;
(ii) control the pressure adjustment module to maintain the internal pressure of the airbag at the first pressure value and, while the displacement sensing module and the measurement site move relative to each other in a direction substantially perpendicular to an orientation of the artery, perform a first distance measuring scan to identify a first measurement position where the vascular radial displacement pulse wave signal output by the displacement sensing module is maximal, the first measurement position corresponding to a location of the artery;
(iii) control the pressure adjustment module to adjust the internal pressure of the airbag based on the first measurement position and to identify a second pressure value when the vascular radial displacement pulse wave signal output by the displacement sensing module is maximal; and
(iv) control the pressure adjustment module to maintain the internal pressure of the airbag at the second pressure value to maintain a pressing depth of the airbag on the first measurement position, thereby enabling the displacement sensing module to perform measurement of the vascular radial displacement pulse wave.

35. The radial displacement pulse wave measuring device of claim 34, wherein at least one of the transparent portion of the housing and the transparent window of the airbag is a transparent plate.

36. The radial displacement pulse wave measuring device of claim 34, further comprising an anti-reflection coating disposed on an outer surface of the transparent window of the airbag.

37. The radial displacement pulse wave measuring device of claim 34, further comprising a reflective layer or a dichroic layer disposed on an inner surface of the contact portion.

38. The radial displacement pulse wave measuring device of claim 34, wherein the displacement sensing module further comprises a filter configured to filter noise from the measurement signal to improve a signal-to-noise ratio of the vascular radial displacement pulse wave signal.

39. The radial displacement pulse wave measuring device of claim 34, wherein the pressure adjustment module comprises a pump and a pulse width modulation circuit for adjusting a rotation speed of the pump.

40. The radial displacement pulse wave measuring device of claim 34, wherein the displacement sensing module comprises a photoelectric displacement sensor.

41. The radial displacement pulse wave measuring device of claim 40, wherein the photoelectric displacement sensor comprises a distance measuring device selected from a group consisting of a laser displacement meter, a fiber-optic displacement sensor, a three-dimensional laser scanner, a time-of-flight (TOF) distance sensor, a three-dimensional time-of-flight (3D TOF) distance array sensor, a laser Doppler anemometer, a laser Doppler velocimeter, a laser Doppler vibrometer, a Michelson interferometer, and a laser interferometer.

42. The radial displacement pulse wave measuring device of claim 34, further comprising a scanning position control module communicatively connected to the computing unit and configured to control the displacement sensing module to automatically perform the first distance measuring scan on the measurement site.

43. The radial displacement pulse wave measuring device of claim 42, wherein the scanning position control module comprises a single axis position controller or a dual axis position controller.
